(19) United States
(12) Patent Application Publication
(10) Pub. No.: US 2006/0003948 A1
(43) Pub. Date: Jan. 5, 2006

(54) Compositions that include a triterpene and a carrier

(76) Inventors: Pavel A. Krasutsky, Duluth, MN (US); Andriy B. Khotkevych, Duluth, MN (US)

Correspondence Address: Schwegman, Lundberg, Woessner & Kluth, P.A., P.O. Box 2938, Minneapolis, MN, 55402, US

(21) Appl. No.: 11/052,451

(22) Filed: Feb. 7, 2005

Related U.S. Application Data
(60) Provisional application No. 60/542,341, filed on Feb. 6, 2004.

(51) Int. Cl. A61K 31/7048; A61K 31/704; A61K 31/57; A61K 31/496; A61K 31/137

(52) U.S. Cl. 514/28; 514/171; 514/559; 514/35; 514/649; 514/49; 514/254.06; 514/254.07

(57) ABSTRACT

The present invention provides for compositions that includes a triterpene (e.g., betulin) and a carrier (e.g., lanolin). The compositions are useful for skin care (e.g., as cosmetic formulations), and as therapeutic formulations, e.g., for treating topical fungal infections.

### RELATED APPLICATIONS

This application claims priority to U.S. Provisional Patent Application Ser. No. 60/542,341, filed Feb. 6, 2004, which is incorporated herein by reference.

### BACKGROUND OF THE INVENTION

Fungi infect humans and are a major cause of human health problems. They also infect plants and cause enormous losses in agricultural productivity. One class of fungal infections of mammals are the dermatophytic infections. These are fungal infections of the hair, nails, and skin. They are caused by fungi called “dermatophytes,” which include species belonging to the genera Epidermophyton, Microsporum, and Trichophyton. Among the species of dermatophytes are the following: Microsporum canis, which results in scalp and skin infections, mostly in children; Microsporum gypseum, which also results in scalp and skin infections in animals and humans; Trichophyton tonsurans, the major agent causing scalp ringworm; Trichophyton rubrum, causing skin, nail, hair, and scalp infections; and Trichophyton mentagrophytes, which can occur on all parts of the body surface. Other fungal infectious agents include the opportunists that are likely to infect immunodeficient persons. These include Cryptococcus, Candida, and Aspergillus.

Outer layers of plants such as leaf cuticles, fruit peels, and bark protect the plant against abrasion, prevent water loss, and protect against pathogenic microorganisms. Breaking through the plant protective outer layer is a prerequisite for a pathogen to enter the plant's internal tissues. Some studies have suggested that penetration of the protective layer involves dissolution of the host cuticle by enzymes secreted by the pathogen. Nicholson, R. L. et al., in The Fungal Spore and Disease Initiation in Plants and Animals, eds. Cole, G. T., and Hoch, H. C., 1991, Plenum Press, New York, pp. 3-23.

Pentacyclic triterpenes are among the most common plant secondary metabolites, but their function in plants has not been fully understood. They are usually concentrated in the outermost layers such as plant cuticle, fruit peel, and bark.

Literature supplies examples of enzymes that can be inhibited by triterpenes, indicating the ability of triterpenes to act broadly in a non-specific mode on multiple targets. For example, Buchler et al. (Biochem. Biophys. Acta 1075, 206 (1991) showed inhibition of rat renal 11β-hydroxysteroid dehydrogenase. Koch et al. (Phytother, Res. 8, 109 (1994)) showed in vitro inhibition of adenosine deaminase. This leads to the hypothesis that pentacyclic triterpenoids in plant protective outer layers may protect against infection by inhibiting enzymes that would degrade the cuticle.

Betulin is a pentacyclic triterpenoid derived from the outer bark of paper birch trees (Betula papyrifera, B. pendula, B. verucosa, etc.). It can be present at concentrations of up to about 24% of the bark of white birch. Merck Index, twelfth edition, page 1236 (1996). Lupeol is a related compound also found in birch bark and in other plant sources. Lupeol is present at concentrations of about 1.5-3% of the birch bark and at up to about 8.2% in Canavalia ensiformis, a plant widespread in the humid tropics of Asia and Africa. Allobetulin is another triterpenoid found in birch bark. A typical pulp mill that process birch produces enough bark waste to allow for the inexpensive isolation of significant quantities of these triterpenoids.

Several triterpenoids have been found to have utility. For example, betulin and related compounds have been shown to have anti-viral activity against herpes simplex virus. Carlson et al., U.S. Pat. No. 5,750,578. Betulin and related compounds have also been shown to have anti-fungal and anti-bacterial activity. However, triterpenoids are hydrophobic compounds with relatively low interfacial activity and water solubility. For instance, the solubility of betulin in water is about 0.15 mg/l. The relatively low interfacial activity and water solubility can make handling and administration of the compounds difficult. Low interfacial activity also limits the efficient interaction with target (fungi or bacteria) cell membranes. It also limits accessibility to hydrophilic biological targets or targets protected by a hydrophilic barrier.

Current agents used to treat fungal infections include the polyene antibiotics, including nystatin; synthetic azoles; and griseofulvin. Fungal infections are difficult to treat because, like humans, they are eukaryotes.

Although many triterpenes have biological activity, the use of triterpenes, particularly for treating plants, presents several drawbacks. Triterpenes dissolve sparingly in water and other aqueous media and thus are difficult to apply to crops in non-emulsion formulations.

Currently, there is a need for new anti-fungal compositions that include triterpenes. The new anti-fungal compositions would include a triterpene in a carrier that could effectively dissolve an effective and safe amount of the triterpene. A need particularly exists for compositions that will act against a range of species, including dermatophytic fungi. New anti-fungal compositions would be less expensive to manufacture if they were abundant natural products or easily synthesized from abundant natural products. As such, the compositions would have biological activity against a range of species, including dermatophytic fungi.

### SUMMARY OF THE INVENTION

The use of a substance selected from the group of lanolin, anhydrous lanolin, acetylated lanolin, lanosterol (lonosta-8,24-dien-3-ol), lanoceric acid, lanocerin, lanocerina, lanochol, lanolin alcohol, lanofier, lanofin, lanogel, lanogene, lanolate, lanolized A, lanolized RC, lanolized WW, liquid lanolin, lanosan, lanosol, lanotex 730, lanoxal 75, lanoxide 52, lanoxide 59, lanoxyl 30, lanpol, lanpolamide, lantox, lantox 55, lantrol, lantrol AWS, lanum, lanotex 730, lanoxal 75, lanoxide 52, lanoxide 59, lanoxyl 30, lanpol, lanidrol, lanigen, laniol, lanisolate, lanpolamide, laneto WWA, lanexol, lanfrax, petrolatum and combinations thereof; effectively dissolves, or assists in dissolving, a therapeutically or cosmetically effective and safe amount of the triterpene, in a therapeutic or cosmetic formulation. As such, the use of a substance selected from the group above (e.g., lanolin) will allow for the presence of relatively larger amounts of a triterpene to be present in the therapeutic or cosmetic formulation.

The compositions described herein can be effective in treating a mammal afflicted with a fungal infection, bacterial infection, viral infection, or a yeast infection. The combination is also effective in inhibiting or killing a fungus, yeast, virus or bacterium. The combination is also effective in skin care (e.g., enhancing skin appearance). The combination is also effective in treating skin afflicted with a condition selected from the group of: a fungal infection, a yeast infection, bacterial infection, viral infection, cracked skin, dry skin, damaged skin, sunburned skin, skin with a minor burn, irritated skin, skin with a rash, chapped skin, raw skin, skin afflicted with Dermatitis, skin with an abrasion, skin associated with Atopic Dermatitis, skin associated with laser resurfacing, skin associated with chemical peels, skin associated with radiation therapy, skin afflicted with acne and treated with Accutane® (Isotretinoin), skin afflicted with erythema, skin afflicted with fissuring, skin with a diaper rash, or a combination thereof.

The present invention provides for new anti-fungal, anti-viral, and/or anti-bacterial compositions that include one or more triterpenes (e.g., betulin, betulinic acid, lupeol, betulin-3-caffeate, allobetulin, or combinations thereof). The composition includes a triterpene in a carrier that effectively dissolves an effective and safe amount of the triterpene. The composition acts against a range of species, including dermatophytic fungi. The compositions can be less expensive to manufacture or can include triterpenes that are easily synthesized from abundant natural products. As such, the compositions could have biological activity against a range of species, including dermatophytic fungi.

The present invention provides a composition that includes:(a) a triterpene (e.g., betulin, betulinic acid, lupeol, betulin-3-caffeate, allobetulin, or combinations thereof); and (b) a substance selected from the group of lanolin, anhydrous lanolin, acetylated lanolin, lanosterol (lonosta-8,24-dien-3-ol), lanoceric acid, lanocerin, lanocerina, lanochol, lanolin alcohol, lanofier, lanofin, lanogel, lanogene, lanolate, lanolized A, lanolized RC, lanolized WW, liquid lanolin, lanosan, lanosol, lanotex 730, lanoxal 75, lanoxide 52, lanoxide 59, lanoxyl 30, lanpol, lanpolamide, lantox, lantox 55, lantrol, lantrol AWS, lanum, lanotex 730, lanoxal 75, lanoxide 52, lanoxide 59, lanoxyl 30, lanpol, lanidrol, lanigen, laniol, lanisolate, lanpolamide, laneto WWA, lanexol, lanfrax, petrolatum and combinations thereof.

The present invention also provides a composition that includes betulin; petrolatum, mineral oil, ceresin and lanolin alcohol.

The present invention also provides a composition that includes betulin; and VASELINE® petroleum jelly.

The present invention also provides a composition that includes betulin; and petroleum jelly.

The present invention also provides a composition that includes betulin; and AQUAPHOR® ointment.

The present invention also provides a composition that includes:(a) a triterpene (e.g., betulin, betulinic acid, lupeol, betulin-3-caffeate, allobetulin, or combinations thereof); and (b) petrolatum (e.g., white petrolatum).

The present invention also provides a therapeutic method for treating a mammal afflicted with a fungal infection or a yeast infection. The method includes administering to a mammal in need of such treatment or at risk thereof, an effective anti-fungal amount of a composition of the present invention.

The present invention also provides a method of inhibiting or killing a fungus or a yeast. The method includes contacting the fungus or yeast with a therapeutically effective anti-fungal or ant-yeast amount of a composition of the present invention.

The present invention also provides a method for enhancing skin appearance. The method includes administering to a skin surface in need of the appearance enhancement a cosmetically effective amount of a composition of the present invention.

The present invention also provides a method for treating skin afflicted with a condition selected from the group of: a fungal infection, a viral infection, a bacterial infection, a yeast infection, cracked skin, dry skin, damaged skin, sunburned skin, skin with a minor burn, irritated skin, skin with a rash, chapped skin, raw skin, skin afflicted with Dermatitis, skin with an abrasion, skin associated with Atopic Dermatitis, skin associated with laser resurfacing, skin associated with chemical peels, skin associated with radiation therapy, skin afflicted with acne and treated with Accutane® (Isotretinoin), skin afflicted with erythema, skin afflicted with fissuring, skin with a diaper rash, or a combination thereof. The method includes administering to a skin surface in need of the treatment or at risk thereof, a composition of the present invention.

The present invention also provides a method of inhibiting or killing a fungus, virus, bacterium or yeast. The method includes contacting the fungus, virus, bacterium or yeast with an effective amount of a composition that includes petrolatum or white petrolatum, mineral oil, ceresin, panthenol, glycerin, polyethylene glycol (PEG), and bisabolol.

The present invention also provides a method of inhibiting or killing a fungus, virus, bacterium or yeast. The method includes contacting the fungus, virus, bacterium or yeast with an effective amount of a composition that includes petrolatum, mineral oil, ceresin and lanolin alcohol.

The present invention also provides a method of inhibiting or killing a fungus, virus, bacterium or yeast. The method includes contacting the fungus, virus, bacterium or yeast with an therapeutically effective amount of a composition that includes VASELINE® petroleum jelly.

The present invention also provides a method of inhibiting or killing a fungus, virus, bacterium or yeast. The method includes contacting the fungus, virus, bacterium or yeast with an therapeutically effective amount of a composition that includes petroleum jelly.

The present invention also provides a method of inhibiting or killing a fungus, virus, bacterium or yeast. The method includes contacting the fungus, virus, bacterium or yeast with an therapeutically effective amount of a composition that includes AQUAPHOR® ointment.

### DETAILED DESCRIPTION OF THE INVENTION

The following definitions are used, unless otherwise described: halo is fluoro, chloro, bromo, or iodo. Alkyl, alkoxy, alkenyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic.

It will be appreciated by those skilled in the art that triterpene compounds present in the compositions of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound present in the compositions of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine antifungal activity using the standard tests described herein, or using other similar tests which are well known in the art.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl;

partially unsaturated (C₂-C₆)alkyl or (C₂-C₆)alkenyl can be vinyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl;

(C₁-C₅)alkanoyl can be carbonyl, acetyl, propanoyl, butanoyl, isopropanoyl, or pentenoyl;

(C₁-C₆)alkoxy can be methoxy, ethoxy, propoxy, isopropoxy, butoxy, iso-butoxy, sec-butoxy, pentoxy, 2-pentoxy, 3-pentoxy, or hexyloxy;

halo(C₁-C₆)alkoxy can be trifluoromethyloxy, 2-chloroethyloxy, 3,3-dichloropropyloxy, or 4,4,4-trifluorobutyloxy;

(C₃-C₈)cycloalkyl can be cyclopropyl, cyclobutyl, cyclopentyl, cyclohexyl, cycloheptyl, or cyclooctyl;

(C₃-C₈)cycloalkyloxy can be cyclopropyloxy, cyclobutyloxy, cyclopentyloxy, cyclohexyloxy, cycloheptyloxy, or cyclooctyloxy;

hydroxy(C₁-C₆)alkoxy can be hydroxymethoxy, 1-hydroxyethoxy, 2-hydroxyethoxy, 1-hydroxypropoxy, 2-hydroxypropoxy, 3-hydroxypropoxy, 1-hydroxybutoxy, 4-hydroxybutoxy, 1-hydroxypentoxy, 5-hydroxypentoxy, 1-hydroxyhexoxy, or 6-hydroxyhexoxy;

amino(C₁-C₆)alkyl can be aminomethyl, 1-aminoethyl, 2-aminoethyl, 1-aminopropyl, 2-aminopropyl, 3-aminopropyl, 1-aminobutyl, 2-aminobutyl, 3-aminobutyl, 4-aminobutyl, 1-aminopentyl, 2-aminopentyl, 3-aminopentyl, 5-aminopentyl, 1-aminohexyl, 2-aminohexyl, 3-aminohexyl, or 6-aminohexyl;

(C₁-C₆)alkoxycarbonyl can be methoxycarbonyl, ethoxycarbonyl, propyloxycarbonyl, isopropyloxycarbonyl, 2-methylpropyloxycarbonyl, butyloxycarbonyl, pentyloxycarbonyl, or hexyloxycarbonyl;

(C₁-C₆)alkanoyloxy can be carbonyloxy, acetyloxy, propanoyloxy, butanoyloxy, 2-methylpropanoyloxy, 2-methylbutanoyloxy, 3-methylbutanoyloxy, pentanoyloxy, or hexanoyloxy.

“N⁺-containing heteroaryl” can be N-pyridinium, N-methyl-2-pyridinium, N-methyl-3-pyridinium, N-methyl-4-pyridinium, N-ethyl-2-pyridinium, N-ethyl-3-pyridinium, N-ethyl-4-pyridinium, 3,5-dimethylpyridinium, or 4-(dimethylamino)pyridinium.

“N⁺-containing heterocycle” can be N-diazabicyclo[2.2.2]octyl; N-azabicyclo[2.2.2]octyl; N-methyl-N-piperidino; N,N-dimethyl-2-piperidino; N,N-dimethyl-3-piperidino; N,N-dimethyl-4-piperidno; N-methyl-N-morpholino; N,N-dimethyl-2-morpholino; or N,N-dimethyl-3-morpholino.

“—N⁺—RₐR_{b}R_{c}” can be N′-benzyl-N,N,N′,N′-tetramethylethylenediamine-N-yl; N,N,N′,N′-tetramethylethylenediamine-N-yl; octyldimethylammonium; tetradecyldimethylammonium; trimethylammonium; triethylammonium, or tri(hydroxymethyl)ammonium.

“3-Carboxypropenoyloxymethyl” refers to the structure —CH₂OC(═O)CH═CHCOOH.

“Aminoacetoxymethyl” refers to the structure —CH₂OC (═O) CH₂NH₂.

“(Carboxymethoxy)acetoxymethyl” refers to the structure —CH₂OC(═O)CH₂OCH₂COOH.

“4-Carboxybutanoyloxymethyl” refers to the structure —CH₂OC (═O) CH₂CH₂CH₂COOH.

“3-Carboxypropanoyloxymethyl” refers to the structure —CH₂OC (═O) CH₂CH₂COOH.

“Carboxycarbonyloxymethyl” refers to the structure —CH₂OC (═O) COOH.

“2-Amino-3-methyl-butanoyloxymethyl” refers to the structure —CH₂OC(═O)CH(NH₂)CH(CH₃)₂.

“4-Carboxy-(3,3-dimethyl)butanoyloxymethyl” refers to the structure —CH₂OC(═O)CH₂C(CH₃)₂CH₂COOH.

“2-Carboxybenzoyloxymethyl” refers to the structure

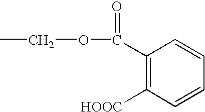

“Butanoyloxymethyl” refers to the structure —CH₂OC(═O)CH₂CH₂CH₃.

“2-Carboxybenzoyl” refers to the structure

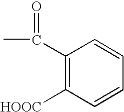

“2-Amino-3-methylbutanoyl” refers to the structure —C(═O)CH₂(NH₂)CH₂(CH₃)₂.

“3-Carboxypropenoyl” refers to the structure —C(═O)CH═CHCOOH.

“Aminoacetyl” refers to the structure —C(═O)CH₂NH₂.

“4-Carboxybutanoyl” refers to the structure —C(═O)CH₂CH₂CH₂COOH.

“(Carboxymethoxy)acetyl” refers to the structure —C(═O)CH₂OCH₂COOH.

“3-(3,4-Dihydroxyphenyl)propenoyl” refers to the structure

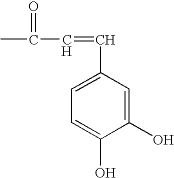

“3-Carboxypropanoyl” refers to the structure —C(═O)CH₂CH₂COOH.

“Carboxycarbonyl” refers to the structure —C(═O)COOH.

“4-Carboxy-(3,3-dimethyl)butanoyl” refers to the structure —C(═O)CH₂C(CH₃)₂CH₂COOH.

“Carboxymethylenethioacetyl” refers to the structure —C(═O)CH₂SCH₂COOH.

“3-Carboxy-3-methylbutanoyl” refers to the structure —C(═O)CH₂C(COOH)(CH₃)₂.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Third Edition, Wiley: N.Y., 1999, and references cited therein). An amino acid can be linked to the remainder of a compound of formula (I)-(VI) through the carboxy terminus, the amino terminus, or through any other convenient point of attachment, such as, for example, through the sulfur of cysteine. The term “peptide” describes a sequence of 2 to 25 amino acids (e.g. as defined hereinabove) or peptidyl residues. The sequence may be linear or cyclic. For example, a cyclic peptide can be prepared or may result from the formation of disulfide bridges between two cysteine residues in a sequence. A peptide can be linked to the remainder of a compound of formula (I)-(VI) through the carboxy terminus, the amino terminus, or through any other convenient point of attachment, such as, for example, through the sulfur of a cysteine. Preferably a peptide comprises 3 to 25, or 5 to 21 amino acids. Peptide derivatives can be prepared as disclosed in U.S. Pat. Nos. 4,612,302; 4,853,371; and 4,684,620.

Glycosides are formed by reacting mono-, di- and polysaccharides with 1-2 hydroxyl groups of the compound of formula (I)-(VI), including glucose, glucuronic acid, mannose, galactose, sorbase, ribose, maltose, sucrose, modified cellulosics, dextrans, modified starches and the like. These derivatives can advantageously exhibit improved water solubility over betulin itself. See, Remington's Pharmaceutical Sciences, A. R. Gennaro, ed., Mack Pub. Co. (18th ed., 1990) at pages 384-386. Glycoside derivatives can be prepared as described in PCT Applications WO 96/34005 and 97/03995.

The term “polyethyleneimine” refers to the group (—NHCH₂CH₂—),[—N(CH₂CH₂NH₂)CH₂CH₂—]_{y}. Polyethyleneimine can be attached to a compound through either of the nitrogen atoms marked with hash marks. “Poly(ethylene glycol)” refers to the compound H(OCH₂CH₂)ₙOH. It can be attached to a compound through its terminal hydroxyl.

The term “partially unsaturated” refers to a linear or branched hydrocarbon having one or more carbon-carbon double bonds.

The term “phosphono” refers to O═P(OH)₂—.

The term “direct bond” refers to a group being absent.

Combinations of substituents and/or variables are permissible only if such combinations result in stable compounds. By “stable compound” is meant herein a compound that is sufficiently robust to survive isolation to a useful degree of purity from a reaction mixture, and formulation into an efficacious antifungal agent.

As used herein, the term “triterpene” can be a plant secondary metabolite that includes a hydrocarbon, or its oxygenated analog, that is derived from squalene by a sequence of straightforward cyclizations, functionalizations, and sometimes rearrangement. Triterpenes or analogues thereof can be prepared by methods known in the art, i.e., using conventional synthetic techniques or by isolation from plants. Suitable exemplary triterpenes and the biological synthesis of the same are disclosed, e.g., in R. B. Herbert, The Biosynthesis of Secondary Plant Metabolites, 2nd. ed. (London: Chapman 1989). The term “triterpene” refers to one of a class of compounds having approximately 30 carbon atoms and synthesized from six isoprene units in plants and other organisms. Triterpenes consist of carbon, hydrogen, and optionally oxygen. Most triterpenes are secondary metabolites in plants. Most, but not all, triterpenes are pentacyclic. The term “triterpene” includes metabolites as well as pro-drugs of the parent compound (i.e., triterpene). Examples of triterpenes include betulin, allobetulin, lupeol, friedelin, and all sterols, including lanosterol, stigmasterol, cholesterol, β-sitosterol, and ergosterol.

“Prodrugs” are intended to include any covalently bonded substances which release the active parent drug or other formulas or compounds of the present invention in vitro or in vivo when, e.g., such prodrug is administered to a mammalian subject. Prodrugs of a compound of the present invention, for example betulin, are prepared by modifying functional groups present in the compound in such a way that the modifications are cleaved, either in routine manipulation in vitro or in vivo, to the parent compound. Prodrugs include compounds of the present invention wherein the hydroxy or amino group is bonded to any group that, when the prodrug is contacted with plant tissue or administered to a mammalian subject, cleaves to form a free hydroxyl or free amino, respectively. Examples of prodrugs include, but are not limited to, acetate, formate and benzoate derivatives of alcohol and amine functional groups in the compounds of the present invention, and the like.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Metabolite” refers to any substance resulting from iochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

As used herein, “treating” or “treat” includes (i) preventing a pathologic condition (e.g., fungal infection) from occurring (e.g. prophylaxis); (ii) inhibiting the pathologic condition (e.g., fungal infection) or arresting its development; and (iii) relieving the pathologic condition (e.g., fungal infection). The term refers to eliminating, preventing or reducing the severity of a symptom associated with a fungal or yeast infection, or the underlying condition itself, such as those described in The Merck Manual, 17^{th} ed. Merck Research Laboratories (1999).

The term “quaternary ammonium salt” refers to a compound comprising at least one positively charged nitrogen atom with four covalent bonds to non-hydrogen atoms. Typically the four bonds will be to carbon atoms. Two or three of the bonds can make up a double or triple bond respectively to a single atom.

The triterpenes present in the compositions of the instant invention also include triterpenes derivatized with N⁺-containing groups. These compounds are found to be rather resistant to hydrolysis. Derivatization with N⁺-containing groups is also found to make the triterpenes present in the compositions of the instant invention rather water soluble. For instance, the solubility of some quaternary salts of betulin disclosed herein is 400-600 g/l.

The term “quaternary ammonium salt of a triterpene” refers to triterpene covalently attached to a group comprising at least one positively charged nitrogen atom with four covalent bonds to non-hydrogen atoms. Examples of quaternary ammonium salts of a triterpene include a compound of formulas (I)-(IV).

The term “fungus” refers to a distinct group of eukaryotic, spore-forming organisms wih absorptive nutrition and lacking chlorophyll. It includes mushrooms, molds, and yeasts.

The term “N-diazabicyclo[2.2.2]octyl” refers to the group

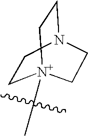

The term “N-pyridinium” refers to the group

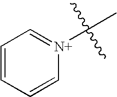

The term “N-methyl-N-piperidino” refers to the group

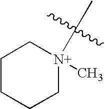

The term “N-methyl-N-morpholino” refers to the group

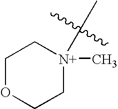

The term “N-azabicyclo[2.2.2]octyl” refers to the group

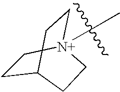

The term “lanolin derivative” or “derivative of lanolin” refers to a chemical and/or physical modification of lanolin, to obtain a substance having suitable properties and characteristics.

The term “humectant” refers to a substance used to obtain a moistening effect. Stedman's Medical Dictionary, Williams & Wilkins, Baltimore, Md., 25^{th} Edition, (1990). A humectant refers to a substance which maintains a moistening effect. Concise Chemical and Technical Dictionary, Chemical Publishing Co., Inc., New York, N.Y., Fourth Enlarged Edition, (1986).

The term “petrolatum” refers to a yellowish mixture of the softer members of the paraffin or methane series of hydrocarbons, obtained from petroleum as an intermediate product in its distillation; typically used as a soothing application to burns and abrasions of the skin, and as a base for ointments. Stedman's Medical Dictionary, Williams & Wilkins, Baltimore, Md., 25^{th} Edition, (1990). Petrolatum refers to a purified mixture of semisolid hydrocarbons. Concise Chemical and Technical Dictionary, Chemical Publishing Co., Inc., New York, N.Y., Fourth Enlarged Edition, (1986).

The term “white petroleum” refers to albolene. Concise Chemical and Technical Dictionary, Chemical Publishing Co., Inc., New York, N.Y., Fourth Enlarged Edition, (1986).

The term “mineral oil” refers to heavy liquid petrolatum; liquid paraffin or petroleum; a mixture of liquid hydrocarbons obtained from petroleum, used as a vehicle in pharmaceutical preparations. Stedman's Medical Dictionary, Williams & Wilkins, Baltimore, Md., 25^{th} Edition, (1990).

The term “ceresin” refers to purified ozokerite; cerin; cerosin; earth wax; mineral wax; a natural mixture of hydrocarbons of high molecular weight; a substitute for beeswax. Stedman's Medical Dictionary, Williams & Wilkins, Baltimore, Md., 25^{th} Edition, (1990).

The term “panthenol” refers (±)2,4-Dihydroxy-N-(3-hydroxypropyl)-3,3-dimethylbutyramide; pantothenyl alcohol. Concise Chemical and Technical Dictionary, Chemical Publishing Co., Inc., New York, N.Y., Fourth Enlarged Edition, (1986).

The term “glycerin” refers to glycerol. Stedman's Medical Dictionary, Williams & Wilkins, Baltimore, Md., 25^{th} Edition, (1990); and Concise Chemical and Technical Dictionary, Chemical Publishing Co., Inc., New York, N.Y., Fourth Enlarged Edition, (1986).

The term “polyethylene glycol (PEG)” refers to a plastic substance made by polymerizing ethylene under pressure; m.w. 1000-3000; m.p. 115-125. Concise Chemical and Technical Dictionary, Chemical Publishing Co., Inc., New York, N.Y., Fourth Enlarged Edition, (1986).

The term “bisabolol” refers to (−)-alpha-bisabolol, alpha-bisabolol, or 4-dimethyl-alpha(4-methyl-3-pentenyl)-3-cyclohexene-1-methanol.

The term “absorption enhancer” refers to a substance that aids in the absorption and/or penetration of another substance, into and/or through the skin membrane.

The term “polyhydric alcohol” refers to an organic compound that contains more than one hydroxyl (OH) group. Concise Chemical and Technical Dictionary, Chemical Publishing Co., Inc., New York, N.Y., Fourth Enlarged Edition, (1986).

The term “skin protectant” refers to a substance that provides a mechanical barrier to guard exposed skin surfaces from harmful or annoying stimuli.

The term “cream” refers to a semisolid emulsion of either the oil-in-water or the water-in-oil type, ordinarily intended for topical use. Stedman's Medical Dictionary, Williams & Wilkins, Baltimore, Md., 25^{th} Edition, (1990).

The term “gel” refers to the solid or semisolid phase of a colloidal solution usually containing medicinal substances and intended for external application. Stedman's Medical Dictionary, Williams & Wilkins, Baltimore, Md., 25^{th} Edition, (1990). The term gel refers to a gelatinous; jelly-like colloid. Concise Chemical and Technical Dictionary, Chemical Publishing Co., Inc., New York, N.Y., Fourth Enlarged Edition, (1986).

The term “ointment” refers to a semisolid preparation usually containing medicinal substances and intended for external application. Bases used as vehicles fall into four general classes: 1) Hydrocarbon bases (oleaginous o. bases) keep medicaments in prolonged contact with the skin, act as occlusive dressings, and are used chiefly for emollient effects. 2) Absorption bases either permit the incorporation of aqueous solutions with the formation of a water-in-oil emulsion or are water-in-oil emulsions that permit the incorporation of additional quantities of aqueous solutions; such bases permit better absorption of some medicaments and are useful as emollients. 3) Water-removable bases (creams) are oil-in-water emulsions containing petrolatum, anhydrous lanolin, or waxes; they may be washed from the skin with water, and are thus more acceptable for cosmetic reasons; they favor absorption of serous discharges in dermatological conditions. 4) Water-soluble bases (greaseless ointment bases) contain only water-soluble substances. Stedman's Medical Dictionary, Williams & Wilkins, Baltimore, Md., 25^{th} Edition, (1990).

The term “lotion” refers to a class of pharmacopeial preparations that are liquid suspensions or dispersions intended for external applications; some consist of finely powdered, insoluble solids held in more or less permanent suspension by suspending agents or surface-active agents, or both; others are oil-in-water emulsions stabilized by surface-active agents. Stedman's Medical Dictionary, Williams & Wilkins, Baltimore, Md., 25th Edition, (1990). The term lotion refers to an aqueous or alcoholic aqueous solution or emulsion for affecting skin. Concise Chemical and Technical Dictionary, Chemical Publishing Co., Inc., New York, N.Y., Fourth Enlarged Edition, (1986).

The term “VASELINE® petroleum jelly” refers to petroleum jelly. Concise Chemical and Technical Dictionary, Chemical Publishing Co., Inc., New York, N.Y., Fourth Enlarged Edition, (1986). The product includes white petrolatum.

The term “petroleum jelly” refers to petrolatum. Stedman's Medical Dictionary, Williams & Wilkins, Baltimore, Md., 25^{th} Edition, (1990).

The term “AQUAPHO®” refers to the petroleum-based ointment commercially available under the trademark AQUAPHOR®. The product includes petrolatum, mineral oil, ceresin, lanolin alcohol, panthenol, glycerin, and bisabolol.

The term “EUCERIN®” refers to the emollient cream product that includes water, petrolatum, mineral oil, ceresin, lanolin alcohol, methylchloroisothiazolinone, and methylisothiazolinone.

The term “plant tissue” refers to a collection of similar cells of a plant, that typically act together to perform a particular function. The term refers to the tissue of any organism of the plant kingdom, as opposed to one of the animal kingdom or of the kingdoms of Fungi, Protista, or Monera. The plant tissue can be any portion or portions of the plant (e.g., bark, roots, leaves, flowers, needles, bulbs, berries, rhizomes, rootstocks, stems, and seeds), as well as the entire plant. The tissues of a plant (“plant tissue”) generally fall into three main categories: dermal tissue, ground tissue, and vascular tissue. Dermal tissue refers to the “skin” layer of all plant organs and is responsible for environmental interaction (light passage, gas exchange, pathogen recognition and protection, color display, etc.). Dermal tissue is composed of epidermal cells, closely packed cells that secrete a waxy cuticle that aids in the prevention of water loss. Ground tissue lies between dermal tissue and vascular tissue. The ground tissue comprises the bulk of the primary plant body. Parenchyma, collenchyma, and sclerenchyma cells are common in the ground tissue. In roots, the ground tissue may store sugars or starches to fuel the spring sap flow; in leaves, the ground tissue is the layer responsible for photosynthesis (the mesophyll). Vascular tissue transports food, water, hormones and minerals within the plant. Vascular tissue includes xylem, phloem, parenchyma, and cambium cells.

As used herein, “bark” refers to the dry, dead outer covering of woody branches, stems and roots of plants that is very distinct and separable from the wood itself. It includes all tissue outside the cambium (growth layer between bark and wood).

As used here the terms “leaf” or “leaves” refer to hose parts of a plant which grow along the sides of ranches or stems or at the bases of plants. Most are reen and contain chlorophyll, though they vary in their shapes and sizes. Leaves are the part of the plant that ordinarily performs photosynthesis (the process that converts sunlight and carbon dioxide into energy).

As used herein, “needle” generally refers to a narrow stiff leaf, such as those of conifers (e.g., pine trees).

As used herein, “root” refers to the part of a plant, normally underground, that absorbs nutrients and anchors the plant into the ground.

As used herein, “bulb” refers to a spheroidal body growing from a plant either above or below the ground (usually below), which is usually a bud, consisting of a cluster of partially developed leaves, and producing, as it grows, a stem above, and roots below, (e.g., the onion or tulip bulb). A true bulb is a complete package containing next year's plant (flower) already forming inside. The contents of the bulb are often enclosed in protective, fleshy scales, which are held together by a small basal plate. The scales are modified leaves that contain enough nutrients to sustain the plant through dormancy and early growth. They may be loose and open like those of a lily, or tightly closed like those of a hyacinth. In many bulbs, a paper-thin tunic protects the scales (lilies don't have a tunic). Roots will grow from the bulb's basal plate.

As used herein, “berry” refers to any small fruit that is pulpy or succulent throughout, having seeds loosely imbedded in the pulp, such as the currant, grape, or blueberry. Berry can be further defined as an indehiscent fruit derived from a single ovary and having the whole wall fleshy, such as the grape or tomato. Furthermore, berries come in various structures including simple, such grape; blueberry, cranberry, or aggregate, such as blackberry; raspberry, strawberry mulberry.

As used herein, “rhizome” refers to a horizontal, usually underground stem that often sends out roots and shoots from its nodes (also called rootstalk or rootstock).

As used herein, “rootstock” refers to a robust plant that provides the root system in grafting, also known as a stock. Scions and buds are grafted and budded to a rootstock or stock. Rootstock also refers to the elongated and often thick rhizomes of certain perennial herbaceous plants such as the Iris, Aspidistra and Solomon's Seal.

As used herein, “stem” refers to the main (usually aerial) axis (sometimes referred to as the trunk or stalk) of a tree, shrub, or plant. “Stem” also refers to the part of the plant that supports the leaves, flowers or fruits of a plant, such as the peduncle of a fruit or the pedicel of a flower.

As used herein, “seed” refers to a ripened ovule, consisting of an embryo with one or more integuments, or coverings, such as an apple seed, a currant seed, dill seed, or kola nut seed. By germination, most seeds produces a new plant. “Seed” also refers to any small seedlike fruit, though it may consist of a pericarp, or even a calyx, as well as the seed proper, such as a parsnip seed or thistle seed. The seed proper has an outer and an inner coat, and within these the kernel or nucleus. The kernel is either the embryo alone, or the embryo enclosed in the albumen, which is the material for the nourishment of the developing embryo. The scar on a seed, left where the stem parted from it, is called the hilum, and the closed orifice of the ovule, the micropyle.

The compositions of the present invention can be used to reduce, treat, or remove the exterior blemishes caused, e.g., by fungal or yeast infections. In particular, the compositions can be used to improve the exterior skin appearance and remove physical signs associated with, e.g., fungal or yeast infections.

As used herein, the term “improve skin appearance” refers to removal or diminishment in appearance or size of skin or nail discolorations and skin blemishes associated, e.g., with fungal or yeast infections.

As used herein, the term “cosmetically effective amount” refers to an amount of a composition of the present invention, sufficient to improve skin appearance.

The term “lanolin” refers to hydrous wool fat; the purified fatlike substance from the wool of sheep, Ovis aries (family Bovidae); it contains not less than 25% and not more than 30% of water; used as a water-adsorbable ointment base. Stedman's Medical Dictionary, Williams & Wilkins, Baltimore, Md., 25^{th} Edition, (1990).

The term “anhydrous lanolin” refers to wool fat that contains not more than 0.25% of water; used as a water-adsorbable ointment base. Stedman's Medical Dictionary, Williams & Wilkins, Baltimore, Md., 25^{th} Edition, (1990). The term anhydrous lanolin refers to yellowish semisolid fat; m.p. 38-42; i.w.; s.eth.; s.bz. Concise Chemical and Technical Dictionary, Chemical Publishing Co., Inc., New York, N.Y., Fourth Enlarged Edition, (1986).

The term “acetylated lanolin” refers to a derivative of lanolin derived from animals. It refers to a hypoallergenic lanolin-derived emollient with a smooth, velvety feel. Acetylated lanolin forms a protective coating on the skin's surface and prevents moisture loss.

The term “lanosterol” refers to (lonosta-8,24-dien-3-ol).

The term “lanoceric acid” refers to C₃₀H₆₀O₄; m.w. 484.780; leaf.; m.p. 104; found combined in wool fat. Concise Chemical and Technical Dictionary, Chemical Publishing Co., Inc., New York, N.Y., Fourth Enlarged Edition, (1986).

The term “lanocerin” refers to waxy lanolin esters. Concise Chemical and Technical Dictionary, Chemical Publishing Co., Inc., New York, N.Y., Fourth Enlarged Edition, (1986).

The term “lanocerina” refers to hydrogenated lanolin. Concise Chemical and Technical Dictionary, Chemical Publishing Co., Inc., New York, N.Y., Fourth Enlarged Edition, (1986).

The term “lanochol” refers to waxy esters and alcohols from lanolin. Concise Chemical and Technical Dictionary, Chemical Publishing Co., Inc., New York, N.Y., Fourth Enlarged Edition, (1986).

The term “lanolin alcohol” refers to the emulsifying and moisturizing agent for moisturizing body lotions, and is commercially available as Amerchol L-101 from Charles Tenant (Toronto, Canada).

The term “lanofier” refers to a lanolin compound. Concise Chemical and Technical Dictionary, Chemical Publishing Co., Inc., New York, N.Y., Fourth Enlarged Edition, (1986).

The term “lanofin” refers to fatty alkanolamide with lanolin. Concise Chemical and Technical Dictionary, Chemical Publishing Co., Inc., New York, N.Y., Fourth Enlarged Edition, (1986).

The term “lanogel” refers to water-soluble lanolin. Concise Chemical and Technical Dictionary, Chemical Publishing Co., Inc., New York, N.Y., Fourth Enlarged Edition, (1986).

The term “lanogene” refers to liquid lanolin esters. Concise Chemical and Technical Dictionary, Chemical Publishing Co., Inc., New York, N.Y., Fourth Enlarged Edition, (1986).

The term “lanolate” refers to ester of lanolin acids. Concise Chemical and Technical Dictionary, Chemical Publishing Co., Inc., New York, N.Y., Fourth Enlarged Edition, (1986).

The term “lanolized A” refers to emulsion of lanolin. Concise Chemical and Technical Dictionary, Chemical Publishing Co., Inc., New York, N.Y., Fourth Enlarged Edition, (1986).

The term “lanolized RC” refers to emulsion of lanolin. Concise Chemical and Technical Dictionary, Chemical Publishing Co., Inc., New York, N.Y., Fourth Enlarged Edition, (1986).

The term “lanolized WW” refers to emulsion of lanolin. Concise Chemical and Technical Dictionary, Chemical Publishing Co., Inc., New York, N.Y., Fourth Enlarged Edition, (1986).

The term “liquid lanolin” refers to vacuum distilled or fractionally solvent extracted lanolin. Concise Chemical and Technical Dictionary, Chemical Publishing Co., Inc., New York, N.Y., Fourth Enlarged Edition, (1986).

The term “lanosan” refers to cationic lanolin derivative. Concise Chemical and Technical Dictionary, Chemical Publishing Co., Inc., New York, N.Y., Fourth Enlarged Edition, (1986).

The term “lanosol” refers to lanolin and mineral oil; nonionic ethylene oxide condensate of natural greases. Concise Chemical and Technical Dictionary, Chemical Publishing Co., Inc., New York, N.Y., Fourth Enlarged Edition, (1986).

The term “lanotex 730” refers to POE lanolin. Concise Chemical and Technical Dictionary, Chemical Publishing Co., Inc., New York, N.Y., Fourth Enlarged Edition, (1986).

The term “lanoxal 75” refers to POE lanolin. Concise Chemical and Technical Dictionary, Chemical Publishing Co., Inc., New York, N.Y., Fourth Enlarged Edition, (1986).

The term “lanoxide 52” refers to POE stearate. Concise Chemical and Technical Dictionary, Chemical Publishing Co., Inc., New York, N.Y., Fourth Enlarged Edition, (1986).

The term “lanoxide 59” refers to POE stearate. Concise Chemical and Technical Dictionary, Chemical Publishing Co., Inc., New York, N.Y., Fourth Enlarged Edition, (1986).

The term “lanoxyl 30” refers to POE lanolin. Concise Chemical and Technical Dictionary, Chemical Publishing Co., Inc., New York, N.Y., Fourth Enlarged Edition, (1986).

The term “lanpol” refers to POE lanolin acids. Concise Chemical and Technical Dictionary, Chemical Publishing Co., Inc., New York, N.Y., Fourth Enlarged Edition, (1986).

The term “lanpolamide” refers to PEG 5 lanolate, PEG 5 lanamide. Concise Chemical and Technical Dictionary, Chemical Publishing Co., Inc., New York, N.Y., Fourth Enlarged Edition, (1986).

The term “lantox” refers to wax from lanolin. Concise Chemical and Technical Dictionary, Chemical Publishing Co., Inc., New York, N.Y., Fourth Enlarged Edition, (1986).

The term “lantox 55” refers to water-dispersible lanolin. Concise Chemical and Technical Dictionary, Chemical Publishing Co., Inc., New York, N.Y., Fourth Enlarged Edition, (1986).

The term “lantrol” refers to oil-soluble liquid fraction of anhydrous lanolin. Concise Chemical and Technical Dictionary, Chemical Publishing Co., Inc., New York, N.Y., Fourth Enlarged Edition, (1986).

The term “lantrol AWS” refers to ethoxylated lantrol. Concise Chemical and Technical Dictionary, Chemical Publishing Co., Inc., New York, N.Y., Fourth Enlarged Edition, (1986).

The term “lanum” refers to purified wool fat; used as ointment base, in cosmetics, leather dressing, rosin soaps. Concise Chemical and Technical Dictionary, Chemical Publishing Co., Inc., New York, N.Y., Fourth Enlarged Edition, (1986).

The term “lanidrol” refers to ethoxylated hydrogenated lanolin. Concise Chemical and Technical Dictionary, Chemical Publishing Co., Inc., New York, N.Y., Fourth Enlarged Edition, (1986).

The term “lanigen” refers to water-attracting substance in wool-fat. Concise Chemical and Technical Dictionary, Chemical Publishing Co., Inc., New York, N.Y., Fourth Enlarged Edition, (1986).

The term “laniol” refers to anhydrous lanolin. Concise Chemical and Technical Dictionary, Chemical Publishing Co., Inc., New York, N.Y., Fourth Enlarged Edition, (1986).

The term “lanisolate” refers to isopropyl lanolate. Concise Chemical and Technical Dictionary, Chemical Publishing Co., Inc., New York, N.Y., Fourth Enlarged Edition, (1986).

The term “laneto WWA” refers to lanolin alcohol. Concise Chemical and Technical Dictionary, Chemical Publishing Co., Inc., New York, N.Y., Fourth Enlarged Edition, (1986).

The term “lanexol” refers to soluble liquid lanolin. Concise Chemical and Technical Dictionary, Chemical Publishing Co., Inc., New York, N.Y., Fourth Enlarged Edition, (1986).

The term “lanfrax” refers to lanolin wax. Concise Chemical and Technical Dictionary, Chemical Publishing Co., Inc., New York, N.Y., Fourth Enlarged Edition, (1986).

Additional lanolin derivatives can be found, e.g., at the Chesham Chemicals website (http://products.cheshamchemicals.co.uk/category2520041. html), dated May 6, 2003; the Total Skin Care website (http://www.totalskincare.com), dated May 6, 2003; the Pharmacosm website (http://www.pharmacosm.it/pdf/CFannin2.pdf), dated May 6, 2003; and the Fancor Personal Care Ingredients website (http://www.fanncorp.com/pc list.htm), dated May 6, 2003.

The term “aloe” refers to the dried juice from the leaves of plants of the genus Aloe (family Liliaceae), from which are derived aloin, resin, emodin, and volatile oils. The dried juice from the leaves of Aloe perryi (socotrine a.'s), of A. barbadensis (Barbados and Curagao a.'s), or of A. capensis (Cape a.'s); used as a purgative. Stedman's Medical Dictionary, Williams & Wilkins, Baltimore, Md., 25^{th} Edition, (1990).

The term “calamine” refers to zinc oxide with a small amount of ferric oxide or basic zinc carbonate suitably colored with ferric oxide; used in dusting powders, lotions, and ointments, as a mild astringent and protective agent for skin disorders. Stedman's Medical Dictionary, Williams & Wilkins, Baltimore, Md., 25^{th} Edition, (1990).

The term “Vitamin E” refers to α-tocopherol; generic descriptor of tocol and tocotrienol derivatives possessing the biological activity of α-tocopherol; contained in various oils (wheat germ, cotton-seed, palm, rice) and whole grain cereals where it constitutes the nonsaponifiable fraction, also in animal tissue (liver, pancreas, heart) and lettuce. Stedman's Medical Dictionary, Williams & Wilkins, Baltimore, Md., 25^{th} Edition, (1990). Concise Chemical and Technical Dictionary, Chemical Publishing Co., Inc., New York, N.Y., Fourth Enlarged Edition, (1986).

The term “Vitamin E acetate” refers to a-tocopherol acetate.

The term “Vitamin C” refers to ascorbic acid. Stedman's Medical Dictionary, Williams & Wilkins, Baltimore, Md., 25^{th} Edition, (1990).

The term “allantoin” refers to glyoxyldiureide; cordianine; 5-ureidohydantoin; a substance present in allontoic fluid, fetal urine, and elsewhere; also an oxidation product of uric acid and the end product of purine metabolism in animals other than man and the other primates. Stedman's Medical Dictionary, Williams & Wilkins, Baltimore, Md., 25^{th} Edition, (1990). The term allantoin refers to cordianine; 5-ureidohydantoin; glyoxyldiureide; C₄H₆N₄O₃; m.w. 158.08; product of animal metabolism excreted in the urine. Concise Chemical and Technical Dictionary, Chemical Publishing Co., Inc., New York, N.Y., Fourth Enlarged Edition, (1986).

The term “aluminum hydroxide gel” refers to a suspension containing Al₂O₃, mainly in the form of aluminum hydroxide, used as an antacid; a dried form, with the same use, is obtained by drying the product of interaction in aqueous solution of an a. salt with ammonium or sodium carbonate. Stedman's Medical Dictionary, Williams & Wilkins, Baltimore, Md., 25^{th} Edition, (1990).

The term “bismuth subnitrate” refers to b. oxynitrate; a basic salt, the composition of which varies with the conditions of preparation; used internally as an intestinal astringent and externally as a mild astringent and antiseptic; the metal is used as an electron microscope stain for nucleic acids. Stedman's Medical Dictionary, Williams & Wilkins, Baltimore, Md., 25^{th} Edition, (1990). The term bismuth subnitrate refers to basic bismuth nitrate; magistery of bismuth; bismuth white; Spanish white. 4BiNO₃(OH)₂BiO(OH) var.; m.w. 1462; odorl. wh. heavy powd.; i.w.; s.HCl; used in enamels, cosmetics, medicine. Concise Chemical and Technical Dictionary, Chemical Publishing Co., Inc., New York, N.Y., Fourth Enlarged Edition, (1986).

The term “boric acid” refers to H₃BO₃; a very weak acid, used as an antiseptic dusting powder, in saturated solution as a collyrium, and with glycerin in aphthae and stomatitis. Stedman's Medical Dictionary, Williams & Wilkins, Baltimore, Md., 25^{th} Edition, (1990). The term boric acid can also refer to sassolite. B₂O₃.3H₂O; m.w. 123.69; tricl.; sp.gr. 1.49; dec. on heating. Concise Chemical and Technical Dictionary, Chemical Publishing Co., Inc., New York, N.Y., Fourth Enlarged Edition, (1986).

The term “cocoa butter” refers to a yellow solid fat obtained from dried kernels of Theobroma cacao bean; sp.gr. 0.976-0.995; m.p. 30-35; i.w.; s.al.; s.chl.; s.eth.; used in making ointments, creams, chocolate. Concise Chemical and Technical Dictionary, Chemical Publishing Co., Inc., New York, N.Y., Fourth Enlarged Edition, (1986).

The term “dimethicone” refers to silicone rubber, silicone latex, latex, dimethyl silicone, simethicone, dimethyl polysiloxane, dermafilm, silbar, dimethicream, poly(dimethylsiloxane), methyl silicone, dimethicone 350, good-rite, gum, hycar, or poly(oxy(dimethylsilylene)); has the molecular formula [—Si(CH3)2O—]n; and the CAS Reg. No.: 9016-00-6.

The term “kaolin” refers to aluminum silicate; powdered and freed from gritty particles by elutriation; used as a demulcent and adsorbent; in dentistry, used to add toughness and opacity to porcelain teeth. Stedman's Medical Dictionary, Williams & Wilkins, Baltimore, Md., 25^{th} Edition, (1990). The term kaolin refers to aluminum silicate; white refractory clay. Concise Chemical and Technical Dictionary, Chemical Publishing Co., Inc., New York, N.Y., Fourth Enlarged Edition, (1986).

The term “pyridoxine hydrochloride” refers to vitamin B6 hydrochloride, has a molecular formula of C8 H11NO3 HCl, and has a CAS Reg. No. of 58-56-0.

The term “shark liver oil” refers to oil obtained from shark liver; red-yel. to lt. br. liq.; sp.gr. 0.91-0.93. Concise Chemical and Technical Dictionary, Chemical Publishing Co., Inc., New York, N.Y., Fourth Enlarged Edition, (1986).

The term “sodium bicarbonate” refers to baking soda; s. acid carbonate; s. hydrogen carbonate; NaHCO₃; used as a gastric systemic antacid, to alkalize urine, and for washes of body cavities. Stedman's Medical Dictionary, Williams & Wilkins, Baltimore, Md., 25^{th} Edition, (1990). The term sodium bicarbonate refers to sodium acid carbonate. Concise Chemical and Technical Dictionary, Chemical Publishing Co., Inc., New York, N.Y., Fourth Enlarged Edition, (1986).

The term “sulfur” refers to Brimstone; an element, symbol S. atomic no. 16, atomic weight 32.066, that combines with oxygen to form s. dioxide (SO₂) and s. trioxide (SO₃), and these with water to make strong acids, and with many metals and non-metallic elements to form sulfides; mildly laxative; has been used externally in the treatment of skin diseases. Stedman's Medical Dictionary, Williams & Wilkins, Baltimore, Md., 25^{th} Edition, (1990). Concise Chemical and Technical Dictionary, Chemical Publishing Co., Inc., New York, N.Y., Fourth Enlarged Edition, (1986).

The term “tannic acid” refers to a tannin, C₇₆H52O₄₆, that occurs in many plants, particularly in the bark of oaks and other members of the Fagaceae; used as a styptic and astringent, and in the treatment of diarrhea; available also as tannic acid glycerite. Sometimes used synonymously with tannin. Stedman's Medical Dictionary, Williams & Wilkins, Baltimore, Md., 25^{th} Edition, (1990). Concise Chemical and Technical Dictionary, Chemical Publishing Co., Inc., New York, N.Y., Fourth Enlarged Edition, (1986).

The term “topical starch” refers to cornstarch.

The term “trolamine” refers to a USAN-approved contraction for triethanolamine, N(CH₂CH₂OH)₃. Stedman's Medical Dictionary, Williams & Wilkins, Baltimore, Md., 25^{th} Edition, (1990). The term trolamine refers to triethanolamine. Concise Chemical and Technical Dictionary, Chemical Publishing Co., Inc., New York, N.Y., Fourth Enlarged Edition, (1986).

The term “zinc acetate” refers to Zn(C₂H₃O₂)2H₂O; an emetic, styptic, and astringent. Stedman's Medical Dictionary, Williams & Wilkins, Baltimore, Md., 25^{th} Edition, (1990). Concise Chemical and Technical Dictionary, Chemical Publishing Co., Inc., New York, N.Y., Fourth Enlarged Edition, (1986).

The term “zinc carbonate” refers to ZnCO₃; m.w. 125.38; col. trig.; sp.gr. 4.44; m.p. —CO₂ 300; i.w.; s.a. Concise Chemical and Technical Dictionary, Chemical Publishing Co., Inc., New York, N.Y., Fourth Enlarged Edition, (1986).

The term “zinc oxide” refers to ZnO; flowers of z.; z. white. Stedman's Medical Dictionary, Williams & Wilkins, Baltimore, Md., 25^{th} Edition, (1990). Concise Chemical and Technical Dictionary, Chemical Publishing Co., Inc., New York, N.Y., Fourth Enlarged Edition, (1986).

The term “zinc sulfate” refers to ZnSo₄.7H₂O; used in the treatment of various skin diseases. Stedman's Medical Dictionary, Williams & Wilkins, Baltimore, Md., 25^{th} Edition, (1990). The term zinc sulfate refers to zinkosite. ZnSO₄. Concise Chemical and Technical Dictionary, Chemical Publishing Co., Inc., New York, N.Y., Fourth Enlarged Edition, (1986).

The term “wax” refers to cera; beeswax; a thick, tenacious substance, plastic at room temperature, secreted by bees for building the cells of their honeycomb. Any substance with physical properties similar to those of beeswax, of animal, vegetable, or mineral origin (oils, lipids, or fats that are solids at room temperature). Esters of high-molecular-weight fatty acids with monohydric or dihydric alcohols (aliphatic or cyclic), that are solid at room temperature. Stedman's Medical Dictionary, Williams & Wilkins, Baltimore, Md., 25^{th} Edition, (1990). The term wax refers to fatty, plastic, amorphous mass of animal, vegetable, mineral, or synthetic origin, usually consisting of higher monohydric alcohol esters of fatty acids or fatty diamides. Concise Chemical and Technical Dictionary, Chemical Publishing Co., Inc., New York, N.Y., Fourth Enlarged Edition, (1986).

The term “beeswax” refers to a natural mixture of crude cerotic acid and myricin; wh. to yel. amor. sld.; sp.gr. 0.965-0.969; m.p. 63-64; s.al.; used in wax polishes, transparent papers, cosmetics, lithographic ink, in medicine. Concise Chemical and Technical Dictionary, Chemical Publishing Co., Inc., New York, N.Y., Fourth Enlarged Edition, (1986).

The structure and carbon numbering of three exemplary compounds present in the compositions of the instant invention are shown below.

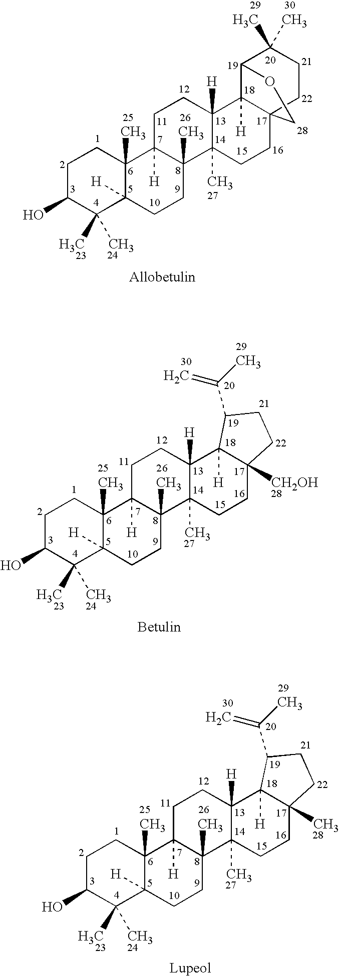

Specific values for compounds of formula (I) are as follows.

A specific value for the bond between carbons 1 and 2 is a single bond.

Another specific value for the bond between carbons 1 and 2 is a double bond.

A specific value for R₁ is hydrogen.

Another specific value for R₁ is hydroxy.

A specific value for R₂ is a direct bond.

A specific value for R₃ is (C₁-C₆) alkyl; wherein any alkyl can optionally be substituted with one or more oxo, carboxy, amino, —OP(═O) (OH)₂, or phenyl; any alkyl is optionally interrupted on carbon with one or more oxy or thio; any alkyl is optionally partially unsaturated; and any aryl can optionally be substituted with one or more hydroxy or carboxy.

Another specific value for R₃ is hydroxymethyl, (carboxymethoxy)acetoxymethyl, 4-arboxybutanoyloxymethyl, 3-carboxypropenoyloxymethyl, 2-carboxybenzoyloxymethyl, 3-carboxypropanoyloxymethyl, aminoacetoxymethyl, carboxycarbonyloxymethyl, 2-amino-3-methyl-butanoyloxymethyl, 4-carboxy-(3,3-dimethyl)butanoyloxymethyl, or —CH₂OC(═O)C(═O)—(—NHCH₂CH₂)ₓ—[—N(CH₂CH₂NH₂)CH₂CH₂]_{y}. A specific value for R₄ is hydrogen or (C₁-C₆)alkyl; wherein any alkyl can optionally be substituted with one or more oxo, carboxy, amino, —OP(═O)(OH)₂, or phenyl; any alkyl is optionally interrupted on carbon with one or more oxy or thio; any alkyl is optionally partially unsaturated; and any aryl can optionally be substituted with one or more hydroxy or carboxy.

Another specific value for R₄ is hydrogen, hydroxymethyl, (carboxymethoxy)acetyl, 4-carboxybutanoyl, 3-carboxypropenoyl, 2-carboxybenzoyl, 3-carboxypropanoyl, aminoacetyl, carboxycarbonyl, 2-amino-3-methyl-butanoyl, 4-carboxy-(3,3-dimethyl)butanoyl, 3-carboxy-3-methylbutanoyl or —C(═O)C(═O)—(—NHCH₂CH₂)—[—N(CH₂CH₂NH₂)CH₂CH₂]_{y}.

A specific value for R₅ is oxy.

A specific group of compounds are compounds of formula (I) wherein R₁ is hydrogen or hydroxy; R₂ is a direct bond; R₃ is (C₁-C₆)alkyl; R₄ is hydrogen or (C₁-C₆)alkyl; and R₅ is oxy or R₄ and R₅ together are oxo; wherein any alkyl can optionally be substituted with one or more oxo, carboxy, amino, or —OP(═O)(OH)₂, or phenyl; any alkyl is optionally interrupted on carbon with one or more oxy or thio; any alkyl is optionally partially unsaturated; and any aryl can optionally be substituted with one or more hydroxy or carboxy.

Another specific group of compounds are compounds of formula (I) wherein R₁ is hydrogen or hydroxy; R₂ is a direct bond; R₃ is hydroxymethyl, (carboxymethoxy)acetoxymethyl, 4-carboxybutanoyloxymethyl, 3-carboxypropenoyloxymethyl, 2-carboxybenzoyloxymethyl, 3-carboxypropanoyloxymethyl, aminoacetoxymethyl, carboxycarbonyloxymethyl, 2-amino-3-methyl-butanoyloxymethyl, 4-carboxy-(3,3-dimethyl)butanoyloxymethyl, or —CH₂OC(═O)C(═O)—(—NHCH₂CH₂)ₓ—[—N(CH₂CH₂NH₂)CH₂CH₂]_{y}; R₄ is hydrogen, hydroxymethyl, (carboxymethoxy)acetyl, 4-carboxybutanoyl, 3-carboxypropenoyl, 2-carboxybenzoyl, 3-carboxypropanoyl, aminoacetyl, carboxycarbonyl, 2-amino-3-methyl-butanoyl, 4-carboxy-(3,3-dimethyl)butanoyl, 3-carboxy-3-methylbutanoyl or —C(═O)C(═O)—(—NHCH₂CH₂)ₓ—[—N(CH₂CH₂NH₂)CH₂CH₂]_{y}; and R₅ is oxy or R₄ and R₅ together are oxo.

Another specific group of compounds of formula (I) is betulin; betulin-3,28-diglycine; betulin-28-glycerol oxalate; betulin-28-glycine; betulin-28-oxalate; betulin arabinose galactan; betulin-3,28-diglycolate; betulin-3-maleate; betulin-3,28-di-(L-glutamic acid γ-benzylester) ester; betulin-3,28-di-L-alanine; betulin-3,28-di-L-proline ester; betulin3,28-dioxalate; betulin-1-ene-2-ol; betulin-3,28-diphenylalanine; betulin-3,28-di-(L-proline ester); betulin-3,28-dioxalate-polyethylene amine; betulin-3,28-diphosphate; betulin-3-caffeate; betulin-3,28-(3′,3′-dimethyl)glutarate; betulin-28-diglycolate; betulin-28-glutarate; betulin-28-maleate; betulin-28-phthalate; betulin-3,28-di(3′,3′-dimethyl) glutarate; betulin-3,28-didiglycolate; betulin-3,28-dithiodiglycolate; betulin-3,28-diglutarate; betulin-3,28-dimaleate; betulin-3,28-diglycolate; betulin-3,28-diphthalate; betulin-3,28-di-L-valine ester; betulin-28-succinate; betulin-3,28-disuccinate; betulin-3,28-di-(polyethylene glycol)-COOH (Mw=1448); betulin-3,28-di-(polyethylene glycol)-COOH (Mw=906 crude); betulin-3,28-di-(polyethylene glycol)-COOH (Mw=906 pure); betulinic acid; betulon-1-ene-2-ol; betulin-3,28-(dipoly(ethylene glycol)bis (carboxymethylester); hederin hydrate; lupeol; lupeol-3-glutarate; lupeol-3-succinate; lupeol-3-thiodiglycolate; lupeol-3-phthalate; oleanolic acid; ursolic acid; or uvaol.

Another specific group of compounds of formula (I) is betulin; betulin-3,28-diglycine; betulin-28-glycerol oxalate; betulin-28-glycine; betulin oxalate; betulin arabinose galactan; betulin-3,28-diglycolate; betulin-3-maleate; betulin di-(L-glutamic acid γ-benzylester) ester; betulin 3,28-di-L-alanine; betulin3,28-di-L-proline; betulin-3,28-dioxalate; betulin-1-ene-2-ol; betulin-3,28-diphenylalanine ester; betulin-3,28-dioxalate-(polyethylene amine); betulin-3-caffeate; betulin-3,28-(3′,3′-dimethyl)glutarate; betulin-28-diglycolate; betulin-28-glutarate; betulin-28-phthalate; betulin-3,28-diglycolate; betulin-3,28-diphthalate; betulin-3,28-phosphate; betulin-28-succinate; betulin-3,28-disuccinate; betulin-3,28-di-(polyethylene glycol)-COOH (Mw=1448); betulin-3,28-di-(polyethylene glycol)-COOH (Mw=906 crude); betulin-3,28-di-(polyethylene glycol)-COOH (Mw=906 pure); betulon-1-ene-2-ol; betulin-3,28-(dipoly(ethylene glycol)bis(carboxymethylester); hederin hydrate; lupeol-3-succinate; lupeol-3-phthalate; lupeol-3-glutarate; oleanolic acid; ursolic acid; or uvaol.

Another specific group of compounds of formula (I) is betulin; betulin-3-maleate; betulin-28-diglycolate; betulin-28-glutarate; betulin-28-maleate; betulin-28-phthalate; betulin-28-succinate; betulin-3,28-diglycine; betulin-3,28-didiglycolate; betulin-3,28-dimaleate; betulin-3,28-dioxalate-3-polyethyleneimine; betulin-3,28-di(3′,3′-dimethyl)glutarate; betulin-3,28-dioxalate-3,28-polyethyleneimine; betulin-3,28-diphthalate; betulin-3,28-disuccinate; betulin-3,28-di-L-valine; lupeol; lupeol-3-amine; lupeol-3-(3′,3′-dimethyl)succinate; lupeol-3-maleate; lupenone; or lupenon-1,2-ene-2-ol.

Specific values for the compounds of formula (II) are as follows.

A specific value for the bond between carbons 1 and 2 is a single bond.

A specific value for R₁ is —O—Y, wherein Y is hydrogen, an amino acid, or (C₁-C₆)alkyl; wherein any alkyl can be optionally substituted with one or more oxo, hydroxy, amino, phenyl, or carboxy any alky can be optionally interrupted with one or more oxy or thio; any phenyl can be optionally substituted with one or more hydroxy or carboxy.

Another specific value for R₁ is —O—Y, wherein Y is hydrogen, 3-carboxypropanoyl, 4-carboxybutanoyl, or 2-amino-2-methylbutanoyl.

A specific value for R₂ is hydrogen.

A specific value for R₃ is hydrogen.

A specific value for R₄ is methyl.

A specific value for R₅ is methyl.

A specific value for R₆ is hydrogen.

A specific value for the bond between carbons 12 and 13 is a single bond.

A specific value for R₇ is hydrogen.

A specific value for R₈ and R₁₁ together is —O—CH₂—.

A specific value for R₉ is methyl.

A specific value for R₁₀ is methyl.

A specific group of compounds of formula (II) is the compounds wherein R₁ is —O—Y and Y is hydrogen, an amino acid, or (C₁-C₆)alkyl; wherein the alkyl of Y can be optionally substituted with one or more oxo, hydroxy, amino, carboxy, or phenyl optionally substituted with one or more hydroxy or carboxy; and can be optionally interrupted with one or more oxy or thio; R₂ is hydrogen; R₃ is hydrogen and the bond between carbons 1 and 2 is a single bond; R₄ and R₅ are each methyl; R₆ is hydrogen and the bond between carbons 12 and 13 is a single bond; R₇ is hydrogen; R₈ and R₁₁ together are —O—CH₂—; and R₉ and R₁₀ are each methyl.

Another specific group of compounds of formula (II) is 3-β-acetoxy-19αH-19,28 lactone oleanan; allobetulin; allobetulin-3-succinate; allobetulin-3-glycine; allobetulin lactone; allobetulin lactone-3-acetate; allobetulin lactone-3-phosphate; allobetulin-3-L-alanine; allobetulin-3-L-valine; allobetulin-3-L-proline ester; allobetulin-3-succinate; allobetulin-3-diglycolate; allobetulin-3-phthalate; allobetulin-3-methylenamine; allobetulin-3-ethanolamine; allobetulin-3-glycolate; allobetulin-3-glutarate; allobetulin-28-glutarate; allobetulin-3-methylamine HCl; allobetulin-3-phosphate; allobetulin-3-(polyethylene glycol)-COOH (Mw=674); allobetulon; allobetulon lactone-1-ene-2-ol; allobetulon lactone-1-en-2-succinate; allobetulon-1-ene-2-ol; allobetulon-1-ene-2-diglycolate; 3-allobetulon-1-ene-2-succinate; allobetulin-3-(poly(ethylene glycol)bis (carboxymethyl ester); or 3-allobetulon-1-ene-2-diglycolate.

Another specific group of compounds of formula (II) is 3-β-acetoxy-19αH-19,28 lactone oleanan; allobetulin; allobetulin-3-succinate; allobetulin lactone; allobetulin lactone-3-acetate; allobetulin lactone-3-phosphate; allobetulin-3-L-valine; allobetulin-3-L-proline; allobetulin-3-succinate; allobetulin-3-diglycolate; allobetulin-3-methylenamine; allobetulin-3-ethanolamine; allobetulin-3-glycolate; allobetulin-3-glutarate; allobetulin-3-glutarate; allobetulin-3-(polyethylene glycol)-COOH (Mw=674); allobetulon; allobetulon lactone-1-ene-2-ol; allobetulon lactone-1-en-2-succinate; allobetulon-1-ene-2-ol; allobetulon-1-ene-2-diglycolate; 3-allobetulon-1-ene-2-succinate; or allobetulin-3-(poly(ethylene glycol)bis(carboxymethyl ester).

Another specific group of compounds of formula (II) is allobetulin, allobetulin-3-glutarate, allobetulin-3-succinate, or allobetulin-3-L-valine.

In one specific embodiment of a compound of formula (IV), R₂, R₅, and R₈ are each independently absent, hydroxyl, N-diazabicyclo[2.2.2]octyl, N-pyridinium, N-alkyl-N-piperidino, N-alkyl-N-morpholino, N-azabicyclo[2.2.2]octyl, or NRₐR_{b}R_{c}; provided at least one of R₂, R₅, and R₈ is N⁺-containing heteroaryl, N⁺-containing heterocycle, or —N⁺RₐR_{b}R_{c}. In this embodiment N-diazabicyclo[2.2.2]octyl; N-pyridinium; N-alkyl-N-piperidino; N-alkyl-N-morpholino; and N-azabicyclo[2.2.2]octyl can optionally be substituted on one or more suitable carbon atoms with one or more oxo, hydroxy, mercapto, alkyl, hydroxyalkyl, halo, nitro, cyano, (C₁-C₆)alkoxy, —COOR_{d}, or —NR_{d}Rₑ. In this embodiment also, any alkyl or alkylene of R₁, R₂, R₄, R₅, R₇, or R₈ can optionally be substituted with one or more oxo or —NR_{d}Rₑ, and optionally interrupted with one or more oxy, imino, or thio, and can optionally be partially unsaturated.

In another specific embodiment of a compound of formula (IV), R₁ is absent and R₂ is hydrogen, N-diazabicyclo[2.2.2]octyl, or N-dimethylamino-N-pyridinium.

In another specific embodiment of a compound of formula (IV), R₃ and R₄ are absent, and R₅ is hydrogen.

In another specific embodiment of a compound of formula (IV), R₃ is oxy; R₄ is absent or (C₁-C₅)alkylenecarbonyl; and R₅ is hydrogen, N-diazabicyclo[2.2.2]octyl; 4-dimethylamino-N-pyridinium; 4-hydroxybutyl-N-diazabicyclo[2.2.2]octyl; 4-benzyl-N-diazabicyclo[2.2.2]octyl; tetramethylethylenediamine-N-yl; N′-benzyl-N,N,N′,N′-tetramethylethylenediamine-N-yl; N-pyridinium; 4-hydroxymethyl-N-pyridinium; 2,4-dimethyl-N-pyridinium; 3,5-dimethyl-N-pyridinium; octyldimethylammonium; or tetradecyldimethylammonium.

In another specific embodiment of a compound of formula (IV), R₆ is oxy; R₇ is absent or (C₁-C₅)alkylenecarbonyl; and R₈ is hydrogen, N-diazabicyclo[2.2.2]octyl; 4-dimethylamino-N-pyridinium; N′-(4-hydroxybutyl)-N-diazabicyclo[2.2.2]octyl; N′-benzyl-N-diazabicyclo[2.2.2]octyl; N,N,N′,N′-tetramethylethylenediamine-N-yl; N′-benzyl-N,N,N′,N′l-tetramethylethylenediamine-N-yl; N-pyridinium; 4-hydroxymethyl-N-pyridinium; 2,4-dimethyl-N-pyridinium; 3,5-dimethyl-N-pyridinium; octyldimethylammonium; tetradecyldimethylammonium; 2-methyl-N-pyridinium; 4-hydroxy-N-methyl-N-piperidinium; or N-methyl-N-morpholino.

In particular embodiments of the invention, the compound of formula (IV) is:

lup-20(29)-ene-3,28-bis-(N-pyridiniumacetate);

lup-20(29)-ene-3-[N-(4-oxybutyl)-1,4-diazabicyclo [2.2.2]octyl-N′-acetate];

lup-20(29)-ene-3,28-bis[N-(1,4-diazabicyclo[2.2.2]octyl)acetate];

lup-20(29)-ene-3,28-bis[N-(N′-benzyldiazabicyclo[2.2.2]octyl)acetate);

lup-20(29)-ene-3,28-bis[N-(N′-(4-oxybutyl)diazabicyclo[2.2.2]octyl)acetate];

lup-20(29)-ene-3-[N-(1,4-diazabicyclo[2.2.2]octyl)acetate];

lup-20 (29)-ene-3,28-bis[(tetramethyletylenediamine-N-yl)acetate];

lup-20(29)-ene-3,28-bis[N′-benzyl-N,N,N′,N′-tetramethylethylenediamine-N-yl)acetate];

lup-20(29)-ene-3-[N-(N′-(benzyl)diazabicyclo[2.2.2]octyl)acetate];

bis(N,N′-pyridinium-2-ethyl)lup-20(29)-ene-3,28-dicarbamate;

1-(3,28-(diacetoxy)lup-20(29)-ene-30-yl)-4-(dimethylamino)pyridinium;

lup-20(29)-ene-3,28-bis(N-pyridinium-2-propionate);

lup-20(29)-ene-3,28-bis(N-pyridinium-3-propionate);

lup-20(29)-ene-3,28-bis(N-pyridinium-4-butyrate);

lup-20(29)-ene-3,28-bis(N-pyridinium-4-butyrate);

lup-20(29)-ene-3,28-bis(N-pyridinium-2-butyrate);

1-[3,28-(diacetoxy)lup-20(29)-ene-30-yl]-1,4-diazabicyclo[2.2.2]octyl;

3,28-bis[3-(1-piperidinyl)propanoyloxy]lup-20(29)-ene;

1-(3,28-dihydroxylup-20(29)ene-30-yl)-4-(dimethylamino)pyridinium;

lup-20(29)-ene-3,28-bis[N-(4-dimethylaminopyridinium)-2-propionate];

lup-20(29)-ene-3,28-bis[N-(1,4-diazabicyclo[2.2.2]octyl)-2-propionate];

1-(lup-20(29)-ene-30-yl)-1,4-diazabicyclo[2.2.2]octane;

1-(3,28-dihydroxylup-20(29)-ene-30-yl)-pyridinium;

lup-20(29)-ene-3,28-bis[N-(1,4-diazabicyclo[2.2.2]octyl)-4-butyrate];

1-(3,28-dihydroxylup-20 (29)-ene-30-yl)-[N-3-(hydroxymethyl)pyridinium];

1-(3,28-dihydroxylup-20(29)-ene-30-yl)-[N-(3,5-dimethylpyridinium)];

bis[N-(1,4-diazabicyclo[2.2.2]octyl)-2-ethyl]-lup-20(29)ene-3,28-idicarbamate;

lup-20(29)-ene-3,28-bis[N-(3-xymethylpyridinium)acetate];

lup-20(29)-ene-3,28-bis[N-(2-oxymethylpyridinium)acetate];

lup-20(29)-ene-3,28-bis[N-(2-methylureapyridinium)acetate];

lup-20(29)-ene-3-(N-(2-oxymethylpyridinium)acetate];

lup-20(29)-ene-3,28-bis[N-(N-methylmorpholino)acetate];

lup-20(29)-ene-3,28-bis[N-(4-hydroxyl-N-methylpiperidino)acetate];

lup-20(29)-ene-3-[N-(3-ureamethylpyridinium)acetate];

lup-20(29)-ene-3-(N-pyridiniumacetate);

lup-20(29)-ene-3,28-bis[N-(1,4-diazabicyclo(2.2.2]octyl)-2-butyrate];

lup-20(29)-ene-3,28-bis[N-(4-dimethylpyridinium)-2-butyrate];

lup-20(29)-ene-3,28-bis[N-(4-dimethylaminopyridinium)-4-butyrate];

lup-20(29)-ene-3,28-bis[N-(4-dimethylaminopyridinium)-3-propionate];

1-(3,28-dihydroxylup-20(29)-ene-30-yl)-4-(hydroxymethyl)pyridinium;

1-(3,28-dihydroxylup-20(29)-ene-30-yl)-3-hydroxy-1-azabicyclo[2.2.2]octane;

lup-20(29)-ene-3,28-bis[N-(2,4-dimethylpyridinium)acetate);

lup-20(29)-ene-3,28-bis[N-(3,5-dimethylpyridinium)acetate];

lup-20(29)-ene-3,28-bis[N-(4-dimethylaminopyridinium)acetate];

lup-20(29)-ene-3-[N-(2-methylpyridinium)acetate];

lup-20(29)ene-3-[N-(2,4-dimethylpyridinium)acetate];

lup-20(29)-ene-3-[N-(4-hydroxy-N-methylpiperidino)acetate];

lup-20(29)-ene-3-[N-(N-methylmorpholino)acetate];

lup-20(29)-ene-3-[N-(3,5-dimethylpyridinium)acetate];

lup-20(29)-ene-3-[N-(4-dimethylaminopyridinium)acetate];

lup-20(29)-ene-3,28-bis(octyldimethylammoniumacetate);

lup-20(29)-ene-3-octyldimethylammoniumacetate;

lup-20(29)-ene-3,28-bis(tetradecyldimethylammoniumacetate);

lup-20(29)-ene-3-tetradecyldimethylammoniumacetate;

N,N,N′,N′-tetramethylethylenediamine-N,N′-bis-[lup-20(29)-ene-3-acetate];

1-[(lup-20(29)-en-3β-yl)oxycarbonylmethyl]-4-aza-1-azonia-bicyclo[2.2.2]octane;

1-[(lup-20(29)-en-3β-yl)oxycarbonylmethyl]trimethylammonium; or

1-[(lup-20 (29) -en-3β-yl)oxycarbonylmethyl]pyridinium.

A specific embodiment of the compound of formula (VI) is the compound wherein R₁ is hydrogen, alkyl, or hydroxyalkyl; R₂ is oxymethylene, thiomethylene, iminomethylene, or methylene; R₃ and R₆ are each independently absent or alkylenecarbonyl; R₄ and R₇ are each independently hydrogen, N-diazabicyclo[2.2.2]octyl; N-pyridinium; N-alkyl-N-piperidino; N-alkyl-N-morpholino; N-azabicyclo[2.2.2]octyl; or NRₐR_{b}R_{c}; or R₁, R₂, R₃, and R₄ are together —O—CH₂—. In this case, N-diazabicyclo[2.2.2]octyl; N-pyridinium; N-alkyl-N-piperidino; N-alkyl-N-morpholino; and N-azabicyclo[2.2.2]octyl can optionally be substituted on carbon with one or more alkyl, hydroxyalkyl, hydroxy, COOR_{d}, or NR_{d}Rₑ. Rₐ, R_{b}, and R_{c} are each independently aryl or (C₁-C₂₄)alkyl; wherein R_{d} and Rₑ are each independently hydrogen or alkyl. Any alkylene or alkyl can optionally be substituted on carbon with one or more oxo, hydroxy, halo, nitro, cyano, trifluoromethyl, COOR_{d}, or —NR_{d}Rₑ, and optionally interrupted with one or more oxy, imino, or thio, and where any alkyl or alkylene can optionally be partially unsaturated.

Another specific embodiment of the compound of formula (VI) is the compound wherein R₁, R₂, R₃, and R₄ are together —O—CH₂—.

Another specific embodiment of the compound of formula (VI) is the compound wherein R₅ is oxy.

Another specific embodiment of the compound of formual (VI) is the compound wherein R₆ is acetyl.

Another specific embodiment of the compound of formual (VI) is the compound wherein R₇ is N-diazabicyclo[2.2.2]octyl; N-pyridinium; or —N⁺(CH₃)₃.

In particular embodiments of the invention, the compound of formula (VI) is:

1-[(19β,28-epoxy-18α-oleanan-3β-yl)oxycarbonylmethyl]-4-aza-1-azonia-bicyclo[2.2.2]octane;

[(19β,28-epoxy-18α-oleanan-3β-yl)oxycarbonylmethyl]trimethylammonium; or

1-[(19β,28-epoxy-18α-oleanan-3β-yl)oxycarbonylmethyl]pyridinium.

A specific class of triterpene compounds present in the compositions of the instant invention include: Betulin; Lupeol; Lupeol acetate; Lupenone; 2-hydroxy-olean-1,2-ene-3-one-28,19-lactone; Allobetulinlactone; Allobetulonlactone; Allobetulinlactone trifluoroacetate; Allobetulinlactone phosphodichloride; 2-brom-Allobetulinlactone; Allobetulinlactone phosphate; Allobetulinlactone acetate; Allobetulin; Allobetulon; Allobetulin trifluoroacetate; Allobetulin phosphodichloride; Allobetulin phosphate; Allobetulin acetate; Allobetulon -1-ene-2-ol; 2-Br-Allobetulin; 3-TMS-O-Allobetulin; 3-aminomethyl-3hydroxy-Allobetulin; Allobetulon cyanohydrin; Allobetulin 3-tosylate; Betulon 28-acetate; Betulin 28-acetate; Betulonic aldehyde; Betulin dimesylate; Betulin-3-O-acetate-28-trifluoroacetate; Betulon; 3-O-acetyl-Betulinic aldehyde; Betulinic aldehyde; Betulon-1-ene-2-ol; Betulin ditrifluoroacetate; Betulin -28-tosylate; Betulin ditosylate; Betulinic acid; Betulonic acid; 3-O-acetyl-Betulinic acid; Betulin caffeate; Betulin dioxalyl chloride; Betulindiamine; Betulin 3-amine; Betulin 28-amine; Betulindihydroxyme; Betulindiphosphate; Betulindiphosphodichloride; Betulindiphosphate sodium salt; Betulin 3,28-bis((1R)-trans-chrysanthemate); Betulin 28-(1R)-trans-chrysanthemate; Betulin bis(N-pyridyl-2-acetate) dichloride; Betulin 3,28-diacrylate; Betulin 3,28-dimethacrylate; Betulin 28-acrylate-3-formiate; Betulin-28-monomethacrylate; Betulin-3,28-bis(P,P′-triphenylphosphinoacetate); Betuline-3,28-bis(tetramethylenediamino acetate); Betuline-3,28-bis(N,N′-diaza[2,2,2]bicyclooctanoacetate); Betulin-3,28-bis(N,N′-dibenzyldiazabicyclo[2.2.2]octanoacetate); Betulin-3,28-bis(N,N′-(4-oxybutyl)diazabicyclo[2.2.2]octanoacetate); Betulin-3,28-bis(oxyacetate); 3,28-Di(methylthiomethylene) betulin; 3-Methylthiomethyleneallobetulin; 28-Methylthiomethylenebetuline 3-acetate; 28-Methylthiomethylenebetul-3-one; Betulin 3-acetate-28-mesylate; Betulin 3,28-di(trifluoroacetamidglycinate); Betulin 28-trifluoroacetamidglycinate; Betulin 3,28-diacetylsalicilate; Betulin 3,28-di(2-oxyethylenoxyoxalate); Allobetulin 3-(poly(ethylene glycol)bis(carboxymethyl)ether)ester; Allobetulin 3-(poly(ethylene glycol)bis(carboxymethyl)ether)methyl ester; Betulin 3,28-di(poly(ethylene glycol)bis(carboxymethyl)ether)ester; Betulin 3,28-di(poly(ethylene glycol)bis(carboxymethyl)ether)ester; Betulin 3,28-di(poly(ethylene glycol)bis(carboxymethyl)ether)methyl ester; Poly(ethylene glycol)bis(carboxymethyl)ether 28,28′ dibetuline ester; Betulin 3,28-di(ethyl) carbamate; Betulin 3,28-disuccinate; Betulin 28-succinate; Betulin 3,28-disuccinyl dipoly(ethylene glycol)ester; 28,28′-Dibetulin poly(propylene glycol) toluene-2,4-dicarbamate terminated; Mixture of suberinic acids; cis-9,10-epoxy-18-hydroxyoctadecanoic acid; cis-9,10-epoxy-18-hydroxyoctadecanoic acid; cis-9,10-epoxy-18-hydroxyoctadecanoic acid+polyethyleneimine; cis-9,10-epoxy-18-hydroxyoctadecanoic acid+polyethyleneimine; cis-9,10-epoxy-18-hydroxyoctadecanoic acid+polyethyleneimine; 22-hydroxydocosanoic acid+polyethyleneimine; Dicarboxylic acids fraction+polyethyleneimine; Potassium salt of cis-9,10-epoxy-18-hydroxyoctadecanoic acid; 22-hydroxydocosanoic acid+polyethyleneimine; Docosandioic acid, 85%+polyethyleneimine; Lupeol 3-(polyethyleneimine propionate); cis-9,10-epoxy-18-hydroxyoctadecanoic acid+polyethyleneimine; Betulin 3,28-disuccinate+polyethyleneimine; Betulin 3,28-disuccinate+polyethyleneimine; Betulin 3,28-disuccinyl polyethyleneimine amide; Betulin 3,28-disuccinyl polyethyleneimine amide; Betulin 3,28-disuccinyl dichloride; Betulin 3,28-disuccinyl (1-methylpyrazine)amide; Lupeol 3-acrylate; cis-9,10-epoxy-18-acetoxyoctadecanoic acid; cis-9,10-epoxy-18-(m-nitrobezoiloxy)octadecanoic acid; cis-9,10-epoxy-18-acetoxyoctadecanoic acid (R)-(+)-α-phenylethylamide; cis-9,10-epoxy-18-(m-nitrobezoiloxy) octadecanoic acid (R)-(+)-α-phenylethylamide; cis-9,10-epoxy-18-hydroxyoctadecanoic acid (1)+polyethyleneimine; cis-9,10-epoxy-18-(3-acetoxylithocholioxy) octadecanoic acid methyl ester; Betulin 3,28-dimaleate+polyethylenimine; Betulin 3,28-dimaleate disodium salt; Betulin 3,28-dimaleate; 9,10,18-trihydroxyoctadecanoic acid; cis-9,10-epoxy-18-hydroxyoctadecanoic acid+polyethyleneimine; Betulin 3,28-diacetate; Betulin 3-acetate; Betulin 3,28-dibenzoate; Betulin 3-benzoate; Betulinic acid methyl ester; Betulin 3,28-di(2′-chloropropionate); Betulin 3,28-di(3′-chloropropionate); Betulin 3,28-di(4′-chlorobutyrate); bis(N,N′-pyridino-2-ethyl) betulin-3,28-carbamate dichloride; Betulin 3,28-di(4′-bromobutyrate); Betulin 3,28-di(2′-bromobutyrate); Betulin-3,28-bis(2-thiuroniumacetate) dihydrochloride; Betulin-3,28-bis (N,N′-pyridino-3-propionate) dichloride; Betulin-3,28-bis(N,N′-pyridino-2-propionate) dichloride; Betulin-3,28-bis(N,N′-pyridino-4-butyrate) dibromide; Betulin -3,28-bis (N,N′-pyridino-4-butyrate) dichloride; Betulin -3,28-bis (N,N′-pyridino-2-butyrate) dibromide; 1-(3,28-diacetoxylup-20-en-30-yl)-4-(dimethylamino) pyridinium bromide; Betulin-3-(N-DABCO-2-acetate); Betulin-3-chloroacetate; Betulin-3(N-benzyl-N′-DABCO-2-acetate); Betulin-3-(N′-oxybutyl-N-DABCO-2-acetate); Mixture of betulin-3-phosphonoacetate and betulin-28-phosphonoacetate; Dihydro-29-carboxy-betulin; Dimethylamide dihydro-29-carboxybetulin; Betulin 3,28-disuccinyl di(4-methyl-4-benzylpyrazonium bromide) amide; 9,10,18-treo-trihydroxyoctadecanoic acid (Phloionolic acid); 22-Hydroxydocosanoic acid (IK32); Birch bark tannin; Birch bark tannin —Na salt; Birch bark tannin —K salt; Betulin-3,28-bis(benzyltetramethyl-ethylenediamino acetate chloride); Betuline-3,28-dioxalate; Betulin-28-maleate; Betulin-3,28-bis(diacetyltartrate); Betulin-3,28-bis(diacetyltartrate) disodium salt; N-(3,28-diacetoxylup-20-en-30-yl)-1,4-diazabicyclo[2.2.2]octane bromide; 3,28,30-triacetoxylup-20(29)-ene; 3,28-bis(3-(1-piperidinyl)propanoyloxy)lup-20(29)-ene dihydrochloride; 30-Bromo-3,28-dihydroxylup-20(29)-ene; 1-(3,28-dihydroxylup-20(29)-en-30-yl)-4-(dimethylamino)pyridinium bromide; 1-(lup-20(29)-en-30-yl)-1,4-diazabicyclo[2.2.2]octane bromide; S-(3,28-dihydroxylup-20(29)-en-30-yl)thiuronium bromide; 1-(3,28-dihydroxylup-20(29)-en-30-yl)-pyridinium bromide; 1-(3,28-dihydroxylup-20(29)-en-30-yl)-3,5-dimethylpyridinium bromide; Adduct of 1 mole of betulin-3-chloroacetate and 1 mole of SV-23; betulin-3,28-bis(2-thiuroniumacetate) dihydrochloride; lup-20(29)-ene-3,28-bis(N,N′-4-dimethylaminopyridino-2-propionate) dichloride; lup-20(29)-ene-3,28-bis(N,N′-1,4-diazabicyclo[2.2.2]octane-2-propionate) dichloride; lup-20(29)-ene-3,28-bis(thiuronium-4-butirate) dichloride; 1-(3,28-dihydroxylup-20(29)-en-30-yl)-4-(hydroxymethyl)pyridinium bromide; 1-(3,28-dihydroxylup-20(29)-en-30-yl)-3-hydroxy-1-azabicyclo[2.2.2]octane bromide; 3,28-dihydroxy-30-(1,2,4-triazol-1-yl)-lup-20(29)-ene; 22-hydroxydocosanoic acid sodium salt; 22-hydroxydocosanoic acid potassium salt; 9,10,18-trihydroxyoctadecanoic acid sodium salt; 9,10,18-trihydroxyoctadecanoic acid potassium salt; 9,10-epoxy-18-hydroxyoctadecanoic acid sodium salt; 9,10-epoxy-18-hydroxyoctadecanoic acid potassium salt; lup-20(29)-ene-3,28-bis(N,N′-1,4-diazabicyclo[2.2.2]octane-4-butyrate) dibromide; lup-20(29)-ene-3,28-bis(N,N′-1,4-diazabicyclo[2.2.2]octane-4-butyrate) dichloride; Bis(N,N′-1,4-diazabicyclo[2.2.2]octane-2-ethyl)-lup-20(29)-ene-3,28-carbamate dichloride; 30-Bromo-3,28-bis(chloroacetyl)lup-20(29)-ene; 1-(3,28-diacetoxylup-20(29)-en-30-yl)-pyridinium bromide; 1-(3,28-dihydroxylup-20(29)-en-30-yl)-3-(hydroxymethyl)pyridinium bromide; lup-20(29)-en-3,28-bis(pyridylmethylurea acetate) dichloride; lup-20(29)-en-3,28-bis(3-oxymethylpyredyniumacetoxy) dichloride; lup-20(29)-en-3,28-bis(2-oxymethylpyredyniumacetoxy) dichloride; lup20(29)-ene-3,28 bis (N,N′-4-dimethylaminopyridino-3-propionate) dichloride; lup20(29)-ene-3,28-bis (N,N′-4-dimethylaminopyridino-4-butyrate) dibromide; lup20(29)-ene-3,28-bis (N,N′-4-dimethylaminopyridino-2-butyrate) dibromide; lup-20(29)-ene-3,28-bis(N,N′-1,4-diazabicyclo[2.2.2]octane-2-butyrate) dibromide; betulin 3-mono(N-pyridyacetate) chloride; lup-20(29)-en-3 mono (2-oxymethylpyredyniumacetoxy) chloride; Betulin 3,28-bis(chloroacetate) dichloride+4-Hydroxy-1-methylpiperidine; Betulin 3,28 bis (chloroacetate) dichloride+4-methylmorpholine; lup-20(29)-en-3 mono(pyridylmethylurea acetate) chloride; 3,28,30-Trihydroxylup-20(29)-ene; Lup20(29)-ene-3,28-bis (2,4-lutidine-1-acetate) dichloride; lup20(29)-ene-3,28-bis(3,5-lutidine-1-acetate) dichloride; lup20(29)-ene-3,28-bis(4-(dimethylamino)-1-(acetate) pyridine) dichloride; lup20(29)-ene-3-(2-Picoline-1-acetate) chloride; lup20(29)-ene-3-mono (2,4-lutidine-1-acetate) chloride; lup20(29)-ene-3(4-hydroxy-1-Methyl, 1-acetate piperidine) chloride; lup20(29)-ene-3 (4′-Methylmorpholine-1-acetate) chloride; lup20(29)-ene-3(3,5-lutidine-1-acetate) chloride; lup20(29)-ene-3(4-(dimethylamino)-1-(acetate)pyridine) chloride; Betulin 3,28 bis(octhyldimethylamoniumacetoxy)dichloride; Betulin 3(octhyldimethylamoniumacetoxy)chloride; Betulin 3,28 bis(tetradecyldimethylamoniumacetoxy)dichloride; Betulin 3 (tetradecyldimethylamoniumacetoxy) chloride; 3,28-dihydroxy-30-(imidazol-1-yl)-lup-20(29)-ene; 3,28-diacetoxy-30-(triazol-1-yl)-lup-20(29)-ene; Betulin-3-(2-chloropropionate); Betulin-3-(N-1-triazolylacetate); Betulin-3-(N-1-triazolyl)-2-propionate; Betulin-3,28-bis(bromoacetate); 3-Acetoxylup-20(29)-ene-28-aldoxyme; 3-Acetoxylup-20(29)-ene-28-aldmethoxyme; Lup-20(29)-ene-3-one-28-al dioxyme; Lup-20(29)-ene-3-one-28-al dimethoxyme; 3-(1,2,4-Triazol-1-yl)acetylallobetulin; 3-(2-(1,2,4-Triazol-1-yl)propionyl)allobetulin; Lup-20(29)-ene-3-acetate-28-p-nitrobenzoate; Lup-20(29)-ene-3-acetate-28-o-nitrobenzoate; Lup-20(29)-ene-3-acetate-28-m-nitrobenzoate; Betulin-3-(N-1-pyrazolyl)-2-propionate; 3,28-bis(2-(triazol-1-yl)propionate)betulin; 28-(2-Chloropropionyl)betulin; 28-(2-(triazol-1-yl)propionyl)betulin; 3,28-bis(2-(imidazol-1-yl)propionyl)betulin; 3,28-Dimethylbetulin; 3-((Imidazol-1-yl)acetoxy)-19β,28-epoxy-18α-oleanan; 3-[2-(Imidazol-1-yl)propionyloxy]-19β,28-epoxy-18α-oleanan; 3-((Pyrazol-1-yl)acetoxy)-19β,28-epoxy-18α-oleanan; 3-[2-(Pyrazol-1-yl)propionyloxy]-19β,28-epoxy-18α-oleanan; 28-(2-imidazolylpropionyloxy) lup-20 (29)-ene; 1-(3,28-dihydroxylup-20(29)-en-30-yl)piperidine; 1-(3,28-diacetoxylup-20(29)-en-30-yl)piperidine; 3,28,30-tris(chloroacetoxy)lup-20(29)-ene; 3β-(N-diazabicyclo[2.2.2]octylacetyloxy)-19β,28-epoxy-18α-oleanan bromide; 3β-(N-diazabicyclo[2.2.2]octylacetyloxy)-19β,28-epoxy-18α-oleanan chloride; 3β-(N-pyridiniumacetyloxy)-19β,28-epoxy-18α-oleanan bromide; 3β-(N-pyridiniumacetyloxy)-19β,28-epoxy-18α-oleanan chloride; 3β-[-(N′,N′-dimethylaminopyridinium)-N-acetyloxy]-19β,28-epoxy-18α-oleanan bromide; 3β-[-(N′,N′-dimethylaminopyridinium)-N-acetyloxy]-19β,28-epoxy-18α-oleanan chloride; 3β-(N-octyldimethylaminoacetyloxy)-19β,28-epoxy-18α-oleanan bromide; 3β-(N-(2-hydroxyethyl)laminoacetyloxy]-19β,28-epoxy-18α-oleanan bromide; 3β-[N,N-dimethyl-N-(2-hydroxyethyl)aminoacetyloxy]-19β,28-epoxy-18α-oleanan bromide; 3β-[N,N-dimethyl-N-(2-hydroxyethyl)aminoacetyloxy]-19β,28-epoxy-18α-oleanan chloride; 3β-[N-(3-hydroxymethylpyridinium)acetyloxy]-19β,28-epoxy-18α-oleanan bromide; 3β-[(N,N,N′,N-′tetramethylethylenediamino)acetyloxy]-19β,28-epoxy-18α-oleanan bromide; 3,28-dimethoxy-30-bromobetulin; combinations thereof; and pharmaceutically acceptable salts thereof.

The compounds present in the compositions of the instant invention can comprise one triterpene moiety derivatized with one or more quaternary ammonium group (e.g., N⁺-containing group). Preferred N⁺-containing groups include N⁺-containing heteraryl, N⁺-containing heterocycle, or —NRₐR_{b}R_{c}, wherein Rₐ, R_{b}, and R_{c} are each independently (C₁-C₂₄)alkyl, aryl, arylalkyl, heteroarylalkyl, heterocycle, or hetercyclealkyl. Preferably, a single triterpene moiety is derivatized with one, two, three, or four N⁺-containing groups.

The compounds present in the compositions of the instant invention can also comprise more than one triterpene moiety derivatized to a single N⁺-containing group and comprise oligomers of alternating triterpene moieties and N⁺-containing groups. In these cases, the triterpene moieties can be further derivatized with additional N⁺-containing groups.

For instance, one embodiment of the invention provides a composition that includes a compound of formula (VII) or (VIII):

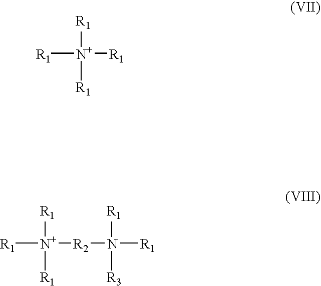

Each R₁ is independently (C₁-C₂₄)alkyl or is alkylcarbonyl attached through the carbonyl to the oxy at the 3 or 28 carbon of betutlin, lupeol, or allobetulin, or to an imino or thio in place of the oxy at the 3 or 28 carbon of betulin, lupeol, or allobetulin, wherein if it is attached to an oxy, imino, or thio at the 28 carbon of allobetulin, carbon 19 is a methylene. R₂ is (C₁-C₂₄)alkyl. R₃ is absent or (C₁-C₂₄)alkyl or is alkylcarbonyl attached through the carbonyl to the oxy at the 3 or 28 carbon of betulin, lupeol, or allobetulin, or to an imino or thio in place of the oxy at the 3 or 28 carbon of betulin, lupeol, or allobetulin, wherein if it is attached to an oxy, imino, or thio at the 28 carbon of allobetulin, carbon 19 is a methylene. Any alkyl or alkylcarbonyl can optionally be substituted with one or more oxo, hydroxy, mercapto, or NR_{d}Rₑ. R_{d} and Rₑ are each independently hydrogen or alkyl. The compound in this case comprises at least two moieties selected from the group of betulin, allobetulin, and lupeol.

In one specific embodiment of the compound of formula (VIII), the compound is N,N,N′,N′-tetramethylethylenediamine-N,N′-bis-[lup-20(29)-ene-3-acetate].

In one embodiment, the compounds present in the compositions of the instant invention include one or more triterpene moieties covalently attached via a linker to a quaternary ammonium salt. The linker can attach to the triterpene moiety at any suitable position of the triterpene. The linker can attach to the quaternary ammonium salt at the N⁺ atom or at any other suitable position. The linker can be, for instance, alkylene, alkylcarbonyl, alkoxy, alkylimino, oxyalkylcarbonyl, carbonylalkylcarbonyl, or carbonylalkyloxy.

The quaternary ammonium salt can also be attached directly to the triterpene without a linker. The attachment in this case can be at any suitable position of the triterpene and any suitable position of the quaternary ammonium salt.

A specific method of the invention is the method of treating a mammal afflicted with a fungal infection comprising administering to the mammal a composition that includes lanolin (or a derivative thereof) and an effective anti-fungal amount of a compound of formula (I)-(VI), wherein the mammal is a human.

Another specific method of the invention is the method of treating a mammal afflicted with a fungal infection comprising administering to the mammal a composition that includes lanolin (or a derivative thereof) and an effective anti-fungal amount of a compound of formula (I)-(VI), wherein the fungal infection is caused by a dermatophytic fungus.

Another specific method of the invention is the method of treating a mammal afflicted with a fungal infection comprising administering to the mammal a composition that includes lanolin (or a derivative thereof) and an effective anti-fungal amount of a compound of formula (I)-(VI), wherein the fungal infection is caused by a dermatophytic fungus that is Microsporum canis, Microsporum gyseum, Microsporum audouinii, Trichophyton tonsurans, Trichophyton mentagrophytes, Epidermophyton floccosum, Trichophyton rubrum, or Pityrosporum ovale.

Another specific method of the invention is the method of treating a mammal afflicted with a fungal infection comprising administering to the mammal a composition that includes lanolin (or a derivative thereof) and an effective anti-fungal amount of a compound of formula (I)-(VI), wherein the fungal infection is caused by Candida albicans or Candida guilliermoundi.

Another specific method of the invention is the method of treating a mammal afflicted with a fungal infection comprising administering to the mammal a composition that includes lanolin (or a derivative thereof) and an effective anti-fungal amount of a compound of formula (I)-(VI), wherein the fungal infection is caused by Biastomyces dermatidis or Cryptococcus neoformans.

Another specific method of the invention is the method of inhibiting or killing a fungus comprising contacting the fungus or yeast with a composition that includes lanolin (or a derivative thereof) and an effective anti-fungal amount of a compound of formula (I)-(VI), wherein the fungus is a dermatophytic fungus.

Another specific method of the invention is the method of inhibiting or killing a fungus comprising contacting the fungus with an effective anti-fungal amount of a composition that includes lanolin (or a derivative thereof) and an effective anti-fungal amount of a compound of formula (I)-(VI), wherein the fungus is a dermatophytic fungus that is Microsporum canis, Microsporum gyseum, Microsporum audouinii, Trichophyton tonsurans, Trichophyton mentagrophytes, Epidermophyton floccosum, Trichophyton rubrum, or Pityrosporum ovale.

Another specific method of the invention is the method of inhibiting or killing a fungus comprising contacting the fungus with an effective anti-fungal amount of a composition that includes lanolin (or a derivative thereof) and an effective anti-fungal amount of a compound of formula (I)-(VI), wherein the fungus is Candida albicans or Candida guilliermoundi.

Another specific method of the invention is the method of inhibiting or killing a fungus comprising contacting the fungus with an effective anti-fungal amount of a composition that includes lanolin (or a derivative thereof) and an effective anti-fungal amount of a compound of formula (I)-(VI), wherein the fungus is Blastomyces dermatidis or Cryptococcus neoformans.

Processes for preparing the triterpenes employed in the invention (i.e., compounds of formula (I)-(VI)) are provided as further embodiments of the invention and are illustrated by the following procedures in which the meanings of the generic radicals are as given above unless otherwise qualified. Specifically, the compounds of formula (I)-(VI) can be prepared from convenient starting materials, employing procedures (e.g., reagents and reaction conditions) known to those of skill in the art. For example, suitable reagents and reaction conditions are disclosed, e.g., in Advanced Organic Chemistry, Part S: Reactions and Synthesis, Second Edition, Carey and Sundberg (1983); Advanced organic Chemistry, Reactions, Mechanisms, and Structure, Second Edition, March (1977); Greene, T. W., Protecting Groups In Organic Synthesis, Third Edition, 1999, New York, John Wiley & sons, Inc.; and Comprehensive Organic Transformations, Second Edition, Larock (1999).

In cases where compounds are sufficiently basic or acidic to form stable nontoxic acid or base salts, administration of the compounds as salts may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compositions that include lanolin (or a derivative thereof) and a compound of formula (I)-(VI) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compositions can be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compositions may be combined with one or more excipients and used in the form of ingestible tablets, buccal tablets, troches, capsules, elixirs, suspensions, syrups, wafers, and the like. Such preparations should contain at least 0.1% of the triterpene compound. The percentage of the compositions can, of course, be varied and may conveniently be between about 2 to about 60% of the weight of a given unit dosage form. The amount of active compound (i.e., triterpene compound) in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound (i.e., triterpene), sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound (i.e., triterpene) may be incorporated into sustained-release preparations and devices.

The composition may also be administered intravenously or intraperitoneally by infusion or injection. Solutions of the triterpene and lanolin (or a derivative thereof) can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the triterpene and lanolin (or a derivative thereof) in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the triterpene and lanolin (or a derivative thereof), plus any additional desired ingredient present in the previously sterile-filtered solutions.

For topical administration, the present compositions may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the triterpene and lanolin (or a derivative thereof) can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compositions of the triterpene and lanolin (or a derivative thereof), to the skin, are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compositions of the triterpene and lanolin (or a derivative thereof) can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compositions of the triterpene and lanolin (or a derivative thereof) in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the triterpene, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The composition is conveniently administered in unit dosage form; for example, containing 5 to 1000 mg, conveniently 10 to 750 mg, most conveniently, 50 to 500 mg of triterpene per unit dosage form.

Ideally, the composition should be administered to achieve peak plasma concentrations of the triterpene of from about 0.5 to about 75 μM, preferably, about 1 to 50 μM, most preferably, about 2 to about 30 μM. This may be achieved, for example, by the intravenous injection of a 0.05 to 5% solution of the triterpene, optionally in saline, or orally administered as a bolus containing about 1-100 mg of the triterpene. Desirable blood levels may be maintained by continuous infusion to provide about 0.01-5.0 mg/kg/hr or by intermittent infusions containing about 0.4-15 mg/kg of the triterpene(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a composition of the invention to act as an anti-fungal agent may be determined using pharmacological models which are well known to the art, including the tests described in the Examples below.

The compositions of the invention may be also be useful as pharmacological tools for the further investigation of the mechanism of their anti-fungal action.

The compositions of the invention can also be administered in combination with other therapeutic agents that are effective to treat fungal infections, or to inhibit or kill a fungus.

The system used to name the triterpenes employed in the compositions of the invention will be clear to one of skill in the art based on the following examples. Names generally consist of the base structure, e.g., betulin, allobetulin, or lupeol, followed by a substituent. For example, betulin-28-succinate, with the structure shown in Example 1, consists of a succinic acid molecule esterified to the hydroxyl at carbon 28 of betulin. If no number is given for the substituent, the substituent is attached to the hydroxyl at carbon 3 on the base structure.

Betulin-3-glycerol oxalate is a compound of formula (I), wherein R₄ and R₅ together are hydrooxyl, R₂ and R₃ together are —OC(═O)C(═O)OCH₂CH(OH)CH₂OH, and R₁ is hydrogen. Betulin-1-ene-2-ol is a compound of formula (I), wherein the bond between carbons 1 and 2 is a double bond, R₁ is hydroxyl, R₂ and R₃ together are hydroxymethyl, and R₄ and R₅ together are oxo. Uvaol is a compound of formula (II) , wherein R₁₀ is methyl, Rg is hydrogen, R₈ is methyl, R₇ is hydrogen, R₁₁ is hydroxymethyl, R₆ is absent and the bond between carbons 12 and 13 is double, R₃ is hydrogen, R₄ and R₅ are methyl, R₂ is hydrogen, and R₁ is hydroxy. Oleanolic acid has the same structure as uvaol, except it has a carboxy at R₁₁ instead of hydroxymethyl. The structure of hederin hydrate is disclosed at page 871 of the Aldrich Chemical Co. 2000-2001 catalog. The structure of other named compounds can be found in standard sources such as the Merck Index. “Betulin arabinose galactan” refers to betulin in a solution of arabino-galactan.

Unless otherwise stated, amino acid substituents are attached to the compounds of the invention through their carboxyl groups via ester linkages. Thus, betulin-3,28-diglycine is the same compound as betulin-3,28-diglycine ester.

The present invention includes the following enumerated embodiments.

### Enumerated Emodiments of the Invention

[1] The present invention provides a composition that includes:

(a) a triterpene, a metabolite thereof, or a pro-drug thereof; and

(b) a substance selected from the group of lanolin, anhydrous lanolin, acetylated lanolin, lanosterol (lonosta-8,24-dien-3-ol), lanoceric acid, lanocerin, lanocerina, lanochol, lanolin alcohol, lanofier, lanofin, lanogel, lanogene, lanolate, lanolized A, lanolized RC, lanolized WW, liquid lanolin, lanosan, lanosol, lanotex 730, lanoxal 75, lanoxide 52, lanoxide 59, lanoxyl 30, lanpol, lanpolamide, lantox, lantox 55, lantrol, lantrol AWS, lanum, lanotex 730, lanoxal 75, lanoxide 52, lanoxide 59, lanoxyl 30, lanpol, lanidrol, lanigen, laniol, lanisolate, lanpolamide, laneto WWA, lanexol, lanfrax, petrolatum, white petrolatum, mineral oil, ceresin, panthenol, glycerin, polyethylene glycol (PEG), bisabolol, and combinations thereof.
[2] The present invention also includes the composition of embodiment [1], wherein the triterpene is a compound of formula (I): 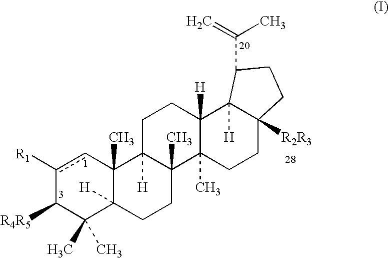
   a metabolite thereof, or a pro-drug thereof, or a pharmaceutically acceptable salt thereof, wherein

R₁ is hydrogen or hydroxy;

R₂ is a direct bond, carbonyl, oxy, thio, carbonyl oxy, oxy carbonyl, (C₆-C₁₀)aryl, or (C₁-C₆)alkyl;

R₃ is hydrogen, hydroxy, hydroxy(C₁-C₆)alkyl, (C₁-C₆)alkyl, O═P(OH)₂, O═P(OH)₂OP(O)(OH)—, (C₁-C₅)alkanoyl, Si(R)₃ wherein each R is H, phenyl or (C₁-C₆)alkyl, C(O)N(R)₂, benzyl, benzoyl, tetrahydropyran-2-yl, 1-[(C₁-C₄)alkoxy](C₁-C₄)alkyl, or a glycoside;

R₄ is hydrogen, hydroxy, hydroxy(C₁-C₆)alkyl, (C₁-C₆)alkyl, O═P(OH)₂, O═P(OH)₂OP(O)(OH)—, (C₁-C₅)alkanoyl, Si(R)₃ wherein each R is H, phenyl or (C₁-C₆)alkyl, C(O)N(R)₂, benzyl, benzoyl, tetrahydropyran-2-yl, 1-((C₁-C₄) alkoxy](C₁-C₄)alkyl, or a glycoside; or R₄ and R₅ together are oxo; and

R₅ is direct bond, carbonyl, oxy, thio, carbonyl oxy, oxy carbonyl, (C₆-C₁₀)aryl, or (C₁-C₆)alkyl; or R₄ and R₅ together are oxo;

wherein any alkyl can optionally be substituted with one or more halo, hydroxy, (C₆-C₁₀)aryl, nitro, cyano, (C₁-C₆)alkoxy, trifluoromethyl, polyethyleneimine, poly(ethylene glycol), oxo, NR₇R₈, wherein R₇ and R₈ are each independently hydrogen, (C₁-C₆)alkyl or polyethyleneimine; or C(═O)OR₉, wherein R₉ is hydrogen, (C₁-C₆)alkyl, or polyethyleneimine;

each of the bonds represented by - - - is independently absent or is present;

wherein any alkyl is optionally interrupted on carbon with one or more oxy, thio, sulfinyl, sulfonyl, polyethyleneimine, or poly(ethylene glycol);

wherein any alkyl is optionally partially unsaturated;

wherein any aryl can optionally be substituted with one or more halo, hydroxy, nitro, cyano, (C₁-C₆)alkoxy, trifluoromethyl, polyethyleneimine, poly(ethylene glycol), oxo, NR₇R₈, wherein R₇ and R₈ are each independently hydrogen, (C₁-C₆)alkyl or polyethyleneimine; or C(═O)OR₉, wherein R₉ is hydrogen, (C₁-C₆)alkyl, or polyethyleneimine.
[3] The present invention also includes the composition of embodiment [2], wherein the bond between carbons 1 and 2 is a single bond.
[4] The present invention also includes the composition of embodiment [2], wherein the bond between carbons 1 and 2 is a double bond.
[5] The present invention also includes the composition of any one of embodiments [2] to [4], wherein R₁ is hydrogen.
[6] The present invention also includes the composition of any one of embodiments [2] to [4], wherein R₁ is hydroxy.
[7] The present invention also includes the composition of any one of embodiments [2] to [6], wherein R₂ is a direct bond.
[8] The present invention also includes the composition of any one of embodiments [2] to [7), wherein R₃ is (C₁-C₆) alkyl; wherein

any alkyl can optionally be substituted with one or more oxo, carboxy, amino, —OP(═O)(OH)₂, or phenyl;

any alkyl is optionally interrupted on carbon with one or more oxy or thio;

any alkyl is optionally partially unsaturated; and

any aryl can optionally be substituted with one or more hydroxy or carboxy.
[9] The present invention also includes the composition of any one of embodiment [8], wherein R₃ is hydroxymethyl, (carboxymethoxy)acetoxymethyl, 4-carboxybutanoyloxymethyl, 3-carboxypropenoyloxymethyl, 2-carboxybenzoyloxymethyl, 3-carboxypropanoyloxymethyl, aminoacetoxymethyl, carboxycarbonyloxymethyl, 2-amino-3-methyl-butanoyloxymethyl, 4-carboxy-(3,3-dimethyl)butanoyloxymethyl, or —CH₂OC(═O)C(═O)—(—NHCH₂CH₂)—[—N(CH₂CH₂NH₂)CH₂CH₂]_{y}.
[10] The present invention also includes the composition of any one of embodiments [2] to [9], wherein R₄ is hydrogen or (C₁-C₆) alkyl; wherein

any alkyl can optionally be substituted with one or more oxo, carboxy, amino, —OP(═O)(OH)₂, or phenyl;

any alkyl is optionally interrupted on carbon with one or more oxy or thio;

any alkyl is optionally partially unsaturated; and

any aryl can optionally be substituted with one or more hydroxy or carboxy.
[11] The present invention also includes the composition of embodiment [10], wherein R₄ is hydrogen, hydroxymethyl, (carboxymethoxy)acetyl, 4-carboxybutanoyl, 3-carboxypropenoyl, 2-carboxybenzoyl, 3-carboxypropanoyl, aminoacetyl, carboxycarbonyl, 2-amino-3-methyl-butanoyl, 4-carboxy-(3,3-dimethyl)butanoyl, 3-carboxy-3-methylbutanoyl or —C(═O)C(═O)—(—NHCH₂CH₂)ₓ—[—N(CH₂CH₂NH₂)CH₂CH₂]_{y}.
[12] The present invention also includes the composition of any one of embodiments [2] to [9], wherein R₅ is oxy.
[13] The present invention also includes the composition of any one of embodiments [2] to [9], wherein R₄ and R₅ together are oxo.
[14] The present invention also includes the composition of embodiment [2] wherein

R₁ is hydrogen or hydroxy;

R₂ is a direct bond;

R₃ is (C₁-C₆)alkyl;

R₄ is hydrogen or (C₁-C₆)alkyl; and

R₅ is oxy or R₄ and R₅ together are oxo; wherein

any alkyl can optionally be substituted with one or more oxo, carboxy, amino, —OP(═O)(OH)₂, or phenyl;

any alkyl is optionally interrupted on carbon with one or more oxy or thio;

any alkyl is optionally partially unsaturated; and

any aryl can optionally be substituted with one or more hydroxy or carboxy.
[15] The present invention also includes the composition of embodiment [2], wherein

R₁ is hydrogen or hydroxy;

R₂ is a direct bond;

R₃ is hydroxymethyl, (carboxymethoxy)acetoxymethyl, 4-carboxybutanoyloxymethyl, 3-carboxypropenoyloxymethyl, 2-carboxybenzoyloxymethyl, 3-carboxypropanoyloxymethyl, aminoacetoxymethyl, carboxycarbonyloxymethyl, 2-amino-3-methyl-butanoyloxymethyl, 4-carboxy-(3,3-dimethyl)butanoyloxymethyl, or —CH₂OC (═O)C(═O)—(—NHCH₂H₂)ₓ—[—N(CH₂CH₂NH₂)CH₂CH₂]_{y};

R₄ is hydrogen, hydroxymethyl, (carboxymethoxy)acetyl, 4-carboxybutanoyl, 3-carboxypropenoyl, 2-carboxybenzoyl, 3-carboxypropanoyl, aminoacetyl, carboxycarbonyl, 2-amino-3-methyl-butanoyl, 4-carboxy-(3,3-dimethyl)butanoyl, 3-carboxy-3-ethylbutanoyl or —C(═O)C(═O)—(—NHCH₂CH₂)ₓ—[—(CH₂CH₂NH₂)CH₂CH₂]_{y}; and

R₅ is oxy or R₄ and R₅ together are oxo.
[16] The present invention also includes the omposition of embodiment [2] wherein the triterpene is etulin; betulin-3,28-diglycine; betulin-28-glycerol xalate; betulin-28-glycine; betulin-28-oxalate; betulin arabinose galactan; betulin-3,28-diglycolate; betulin-3-maleate; betulin-3,28-di-(L-glutamic acid γ-benzylester) ester; betulin-3,28-di-L-alanine; betulin-3,28-di-L-proline ester; betulin-3,28-dioxalate; betulin-1-ene-2-ol; betulin-3,28-diphenylalanine; betulin-3,28-dioxalate-polyethylene amine; betulin-3,28-diphosphate; betulin-3-caffeate; betulin-3,28-(3′,3′-dimethyl)glutarate; betulin-28-diglycolate; betulin-28-glutarate; betulin-28-maleate; betulin-28-phthalate; betulin-3,28-di(3′,3′-dimethyl) glutarate; betulin-3,28-didiglycolate; betulin-3,28-dithiodiglycolate; betulin-3,28-diglutarate; betulin-3,28-dimaleate; betulin-3,28-diglycolate; betulin-3,28-diphthalate; betulin-3,28-di-L-valine ester; betulin-28-succinate; betulin-3,28-disuccinate; betulin-3,28-di-(polyethylene glycol)-COOH (Mw=1448); betulin-3,28-di-(polyethylene glycol)-COOH (Mw=906); betulin-3,28-di-(polyethylene glycol)-COOH (Mw=906); betulinic acid; betulon-1-ene-2-ol; betulin-3,28-(dipoly(ethylene glycol)bis (carboxymethylester); hederin hydrate; lupeol; lupeol-3-glutarate; lupeol-3-succinate; lupeol-3-thiodiglycolate; lupeol-3-phthalate; oleanolic acid; ursolic acid; uvaol; betulin oxalate; betulin di-(L-glutamic acid γ-benzylester) ester; betulin3,28-di-L-proline; betulin-3,28-diphenylalanine ester; betulin-3,28-phosphate; betulin-3,28-dioxalate-3-polyethyleneimine; betulin-3,28-di(3′,3′-dimethyl)glutarate; betulin-3,28-dioxalate-3,28-polyethyleneimine; betulin-3,28-di-L-valine; lupeol-3-amine; lupeol-3-(3′,3′-dimethyl)succinate; lupeol-3-maleate; lupenone; or lupenon-1,2-ene-2-ol.
[17] The present invention also includes the composition of embodiment [1], wherein the triterpene is a compound of formula (II): 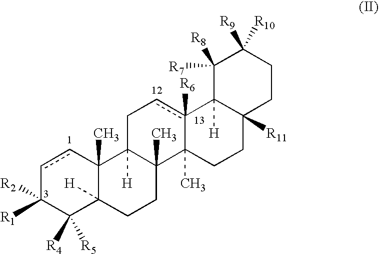
   a metabolite thereof, or a pro-drug thereof, or a pharmaceutically acceptable salt thereof wherein

one of R₁ and R₂ is —O—Y and the other is hydrogen or (C₁-C₆)alkyl optionally substituted by hydroxy, (C₁-C₆)alkoxy, halo, halo(C₁-C₆)alkoxy or NRⱼRₖ wherein Rⱼ and Rₖ are independently H, (C₁-C₆)alkyl or (C₁-C₆)alkonyl; or R₁ and R₂ together are oxo (═O);

R₃ is hydrogen, halo, carboxy, mercapto, (C₁-C₆)alkyl, (C₃-C₈)cycloalkyl, or —O—Y;

R₄ and R₅ are each independently hydrogen, (C₁-C₆)alkyl, or hydroxy(C₁-C₆)alkyl;

R₆ is hydrogen or is absent when the adjacent - - - is a bond;

R₇ is hydrogen or (C₁-C₆) alkyl;

R₈ is hydrogen, (C₁-C₆)alkyl, or hydroxy(C₁-C₆)alkyl and R₁₁ is hydrogen, (C₁-C₆)alkyl, carboxy, or hydroxy(C₁-C₆) alkyl; or R₈ and R₁₁ together are —O—C(═X)—;

R₉ and R₁₀, are each independently hydrogen or (C₁-C₆) alkyl;

each of the bonds represented by - - - is independently absent or is present;

X is two hydrogens, oxo (═O) or thioxo (═S);

each Y is independently H, aryl, P(O) (Cl)₂, (C₃-C₈)cycloalkyl, adamantyl, —SO₂Rₐ O═P(R_{b})₂, O═P(R_{c})₂OP(O)(R_{d})—, Si(Re)₃, tetrahydropyran-2-yl, an amino acid, a peptide, a glycoside, or a 1 to 10 membered branched or unbranched carbon chain optionally that includes 1, 2, or 3 heteroatoms selected from non-peroxide oxy, thio, and —N(R_{f})—; wherein said chain may optionally be substituted on carbon with 1, 2, 3, or 4 oxo (═O), hydroxy, carboxy, halo, mercapto, nitro, —N(R_{g})(Rₕ), (C₃-C₈) cycloalkyl, (C₃-C₈)cycloalkyloxy, aryl, aryloxy, adamantyl, adamantyloxy, hydroxyamino, trifluoroacetylamino, a glycoside, an amino acid, or a peptide; and wherein said chain may optionally be saturated or unsaturated (e.g. containing one, two, three or more, double or triple bonds);

Rₐ is (C₁-C₆)alkyl or aryl;

R_{b}, R_{c}, and R_{d} are each independently hydroxy, (C₁-C₆)alkoxy, hydroxy(C₂-C₆)alkoxy, adamantyloxy, adamantyl(C₁-C₆)alkoxy, norbornyloxy, 1,1-di(hydroxymethyl)-2-hydroxyethoxy, carboxy(C₁-C₆)alkoxy, 2,3-epoxypropyloxy, benzyloxy, (C₃-C₈) cycloalkyloxy, NRₓR_{y}, or aryloxy;

Rₑ is H, aryl or (C₁-C₆)alkyl;

R_{f} is hydrogen, (C₁-C₆)alkyl, (C₁-C₆)alkanoyl, phenyl or benzyl;

R_{g} and Rₕ are each independently selected from the group consisting of hydrogen, (C₁-C₆)alkyl, hydroxy(C₁-C₆)alkyl, adamantyl, adamantyl(C₁-C₆)alkyl, amino(C₁-C₆)alkyl, aminosulfonyl, (C₁-C₆)alkanoyl, aryl and benzyl; or R_{b} and R_{c} together with the nitrogen to which they are attached form a pyrrolidino, piperidino, or morpholino radical; and

Rₓ and R_{y} are each independently hydrogen, (C₁-C₆)alkyl, (C₁-C₆)alkanoyl, aryl or benzyl;

wherein each aryl of Y, Rₐ—R_{d}, R_{g}—Rₕ, Rₓ, and R_{y} may optionally be substituted by 1, 2, or 3 aminosulfonyl, carboxy, NRᵢRⱼ, (C₁-C₆)alkyl, (C₁-₆)alkoxy, hydroxy, halo, nitro, cyano, mercapto, carboxy, hydroxy(C₁-C₆)alkyl, halo(C₁-C₆)alkyl, trifluoromethoxy, (C₁-C₆)alkanoyl, (C₁-C₆)alkoxycarbonyl, (C₁-C₆)alkylthio, or (C₁-C₆)alkanoyloxy; wherein Rᵢ and Rⱼ are each independently hydrogen, (C₁-C₆)alkyl, (C₁-C₆)alkanoyl, phenyl, or benzyl;

wherein any alkyl can optionally be substituted with one or more polyethyleneimine or poly(ethylene glycol); and wherein any alkyl can optionally be interrupted with one or more polyethyleneimine or poly(ethylene glycol).
[18] The present invention also includes the composition of embodiment [17], wherein the bond between carbons 1 and 2 is a single bond.
[19] The present invention also includes the composition of any one of embodiments [17] to [18], wherein R₁ is —O—Y and Y is hydrogen, an amino acid, or (C₁-C₆) alkyl; wherein

any alkyl can be optionally substituted with one or more oxo, hydroxy, amino, phenyl, or carboxy

any alky can be optionally interrupted with one or more oxy or thio;

any phenyl can be optionally substituted with one or more hydroxy or carboxy.
[20] The present invention also includes the composition of any one of embodiments [17] to [18], wherein R₁ is —O—Y and Y is hydrogen, 3-carboxypropanoyl, 4-carboxybutanoyl, or 2-amino-2-methylbutanoyl.
[21] The present invention also includes the composition of any one of embodiments [17] to [20], wherein R₂ is hydrogen.
[22] The present invention also includes the composition of any one of embodiments [17] to [21], wherein R₃ is hydrogen.
[23] The present invention also includes the composition of any one of embodiments [17] to [22], wherein R₄ is methyl.
[24] The present invention also includes the composition of any one of embodiments [17] to [23], wherein R₅ is methyl.
[25] The present invention also includes the composition of any one of embodiments [17] to [24], wherein R₆ is hydrogen and the bond between carbons 12 and 13 is a single bond.
[26] The present invention also includes the composition of any one of embodiments [17] to [25], wherein R₇ is hydrogen.
[27] The present invention also includes the composition of any one of embodiments [17] to [26], wherein R₈ and R₁₁ together are —O—CH₂—.
[28] The present invention also includes the composition of any one of embodiments [17] to [27], wherein R₉ is methyl.
[29] The present invention also includes the composition of any one of embodiments [17] to [28], wherein R₁₀ is methyl.
[30] The present invention also includes the composition of embodiment [17], wherein:

R₁ is —O—Y and Y is hydrogen, an amino acid, or (C₁-C₆) alkyl; wherein

the alkyl of Y can be optionally substituted with one or more oxo, hydroxy, amino, carboxy, or phenyl optionally substituted with one or more hydroxy or carboxy;

and can be optionally interrupted with one or more oxy or thio;

R₂ is hydrogen;

R₃ is hydrogen and the bond between carbons 1 and 2 is a single bond;

R₄ and R₅ are each methyl;

R₆ is hydrogen and the bond between carbons 12 and 13 is a single bond;

R₇ is hydrogen

R₈ and R₁₁ together are —O—CH₂—; and

R₉ and R₁₀ are each methyl.
[31] The present invention also includes the composition of embodiment [17], wherein the triterpene is: 3-β-acetoxy-19αH-19,28 lactone oleanan; allobetulin; allobetulin-3-succinate; allobetulin-3-glycine; allobetulin lactone; allobetulin lactone-3-acetate; allobetulin lactone-3-phosphate; allobetulin-3-L-alanine; allobetulin-3-L-valine; allobetulin-3-L-proline; allobetulin-3-succinate; allobetulin-3-diglycolate; allobetulin-3-phthalate; allobetulin-3-methylenamine; allobetulin-3-ethanolamine; allobetulin-3-glycolate; allobetulin-3-glutarate; allobetulin-28-glutarate; allobetulin-3-methylamine HCl; allobetulin-3-phosphate; allobetulin-3-(polyethylene glycol)-COOH (Mw=674); allobetulon; allobetulon lactone 1-ene-2-ol; allobetulon lactone-1-en-2-succinate; allobetulon-1-ene-2-ol; allobetulon-1-ene-2-diglycolate; 3-allobetulon-1-ene-2-succinate; allobetulin-3-(poly(ethylene glycol)bis (carboxymethyl ester); or 3-allobetulon-1-ene-2-diglycolate.
[32] The present invention also includes the composition of embodiment [1], wherein the triterpene is a quaternary ammonium salt of a triterepene.
[33] The present invention also includes the composition of embodiment [1], wherein the triterpene is a compound of formula (III): 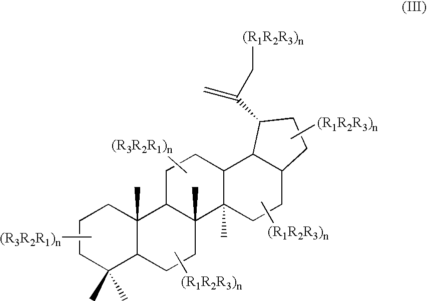
   a metabolite thereof, or a pro-drug thereof, or a pharmaceutically acceptable salt thereof wherein

each R₁ is independently absent, oxy, thio, or imino;

each R₂ is independently absent or alkylene;

each R₃ is independently hydrogen, N⁺-containing heteroaryl, N⁺-containing heterocycle, or —N⁺RₐR_{b}R_{c}; provided at least one R₃ is N⁺-containing heteroaryl, N⁺-containing heterocycle, or —N⁺RₐR_{b}R_{c};

wherein Rₐ, R_{b}, and R_{c} are each independently (C₁-C₂₄)alkyl, aryl, arylalkyl, heteroarylalkly, heterocycle, or heterocylealkyl;

wherein each n is independently 0-4, provided at least one n is not 0;

wherein any heteroaryl, heterocycle, or Rₐ, R_{b}, or R_{c} of R₃ can optionally be substituted on carbon with one or more alkyl, hydroxyalkyl, arylalkyl, heteroarylalkyl, aryl, heterocycle, heterocyclealkyl, oxo, hydroxy, halo, nitro, cyano, (C₁-C₆)alkoxy, trifluoromethyl, —COOR_{d}, —NR_{d}Rₑ, or cycloalkylalkyl;

wherein any cycloalkylalkyl can optionally be substituted on carbon with one or more hydroxyl, N⁺-containing heteroaryl, N⁺-containing heterocycle, or —N+RₐR_{b}R_{c} N⁺-containing heteroarylalkyloxy, N⁺-containing heterocyclealkyloxy, or —N⁺RₐR_{b}R_{c}oxy;

wherein R_{d} and Rₑ are each independently hydrogen or alkyl;

wherein any alkyl or alkylene of R₃ can optionally be substituted on carbon with one or more oxo, hydroxy, halo, nitro, cyano, (C₁-C₆)alkoxy, trifluoromethyl, —COOR_{d}, or —NR_{d}Rₑ, and optionally interrupted on carbon with one or more oxy, imino, or thio, and is optionally partially unsaturated.
[34] The present invention also includes the composition of embodiment [1], wherein the triterpene is a compound of formula (IV): 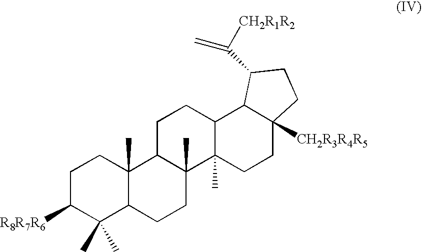
   a metabolite thereof, or a pro-drug thereof, or a pharmaceutically acceptable salt thereof wherein

R₁, R₄, and R₇ are each independently absent or alkylene;

R₃ and R₆ are each independently absent, oxy, thio, or imino;

R₂, R₅, and R₈ are each independently hydrogen, N⁺-containing heteroaryl, N⁺-containing heterocycle, or —N⁺RₐR_{b}R_{c}; provided at least one of R₂, R₅, and R₈ is N⁺-containing heteroaryl, N⁺-containing heterocycle, or —N⁺RₐR_{b}R_{c};

wherein Rₐ, R_{b}, and R_{c} are each independently (C₁-C₂₄)alkyl, aryl, arylalkyl, heteroarylalkly, heterocycle, or heterocylealkyl;

wherein any heteroaryl, heterocycle, Rₐ, R_{b}, or Rc of R₂, R₅, and R₈ can optionally be substituted on carbon with one or more alkyl, hydroxyalkyl, arylalkyl, heteroarylalkyl, aryl, heterocycle, heterocyclealkyl, oxo, hydroxy, halo, nitro, cyano, (C₁-C₆)alkoxy, trifluoromethyl, —COOR_{d}, —NR_{d}Rₑ, or cycloalkylalkyl;

wherein any cycloalkylalkyl can optionally be substituted on carbon with one or more hydroxyl, N⁺-containing heteroaryl, N⁺-containing heterocycle, —N⁺RₐR_{b}R_{c}, N⁺-containing heteroarylalkyloxy, N⁺-containing heterocyclealkyloxy, or —N⁺RₐR_{b}R_{c}oxy;

wherein R_{d} and Rₑ are each independently hydrogen or alkyl;

wherein any alkyl or alkylene of R₁, R₂, R₄, R₅, R₇, or R₈ can be optionally substituted on carbon with one or more oxo, hydroxy, halo, nitro, cyano, (C₁-C₆)alkoxy, trifluoromethyl, —COOR_{d}, or —NR_{d}Rₑ, and optionally interrupted on carbon with one or more oxy, imino, or thio, and is optionally partially unsaturated.
[35] The present invention also includes the composition of embodiment [34], wherein R₂, R₅, and R₈ are each independently absent, hydroxyl, N-diazabicyclo[2.2.2]octyl, N-pyridinium, N-alkyl-N-piperidino, N-alkyl-N-morpholino, N-azabicyclo[2.2.2]octyl, or —NRₐR_{b}R_{c}; provided at least one of R₂, R₅, and R₈ is N⁺-containing heteroaryl, N⁺-containing heterocycle, or —N⁺RₐR_{b}R_{c};

wherein N-diazabicyclo[2.2.2]octyl; N-pyridinium; N-alkyl-N-piperidino; N-alkyl-N-morpholino; and N-azabicyclo[2.2.2]octyl can optionally be substituted on one or more suitable carbon atoms with one or more oxo, hydroxy, mercapto, alkyl, hydroxyalkyl, halo, nitro, cyano, (C₁-C₆)alkoxy, —COOR_{d}, or —NR_{d}Rₑ;

wherein any alkyl or alkylene of R₁, R₂, R₄, R₅, R₇, or R₈ can optionally be substituted with one or more oxo or —NR_{d}Rₑ, and optionally interrupted with one or more oxy, imino, or thio, and can optionally be partially unsaturated.
[36] The present invention also includes the composition of embodiment [34], wherein R₁ is absent and R₂ is hydrogen, N-diazabicyclo[2.2.2]octyl, or N-dimethylamino-N-pyridinium.
[37] The present invention also includes the composition of embodiment [34], wherein R₃ and R₄ are absent, and R₅ is hydrogen.
[38] The present invention also includes the composition of embodiment [34], wherein

R₃ is oxy;

R₄ is absent or (C₁-C₅)alkylenecarbonyl; and

R₅ is hydrogen, N-diazabicyclo[2.2.2]octyl; 4-dimethylamino-N-pyridinium; 4-hydroxybutyl-N-diazabicyclo[2.2.2]octyl; 4-benzyl-N-diazabicyclo[2.2.2]octyl; tetramethylethylenediamine-N-yl; N′-benzyl-N,N,N′,N′-tetramethylethylenediamine-N-yl; N-pyridinium; 4-hydroxymethyl-N-pyridinium; 2,4-dimethyl-N-pyridinium; 3,5-dimethyl-N-pyridinium; octyldimethylammonium; or tetradecyldimethylammonium.
[39] The present invention also includes the composition of embodiment [34], wherein:

R₆ is oxy;

R₇ is absent or (C₁-C₅)alkylenecarbonyl; and

R₈ is hydrogen, N-diazabicyclo[2.2.2]octyl; 4-dimethylamino-N-pyridinium; N′-(4-hydroxybutyl)-N-diazabicyclo[2.2.2]octyl; N′-benzyl-N-diazabicyclo[2.2.2]octyl; N,N,N′,N′-tetramethylethylenediamine-N-yl; N′-benzyl-N,N,N′,N′-tetramethylethylenediamine-N-yl; N-pyridinium; 4-hydroxymethyl-N-pyridinium; 2,4-dimethyl-N-pyridinium; 3,5-dimethyl-N-pyridinium; octyldimethylammonium; tetradecyldimethylammonium; 2-methyl-N-pyridinium; 4-hydroxy-N-methyl-N-piperidinium; or N-methyl-N-morpholino.
[40] The present invention also includes the composition of embodiment [1], wherein the triterpene is:

lup-20(29)-ene-3,28-bis-(N-pyridiniumacetate);

lup-20(29)-ene-3-[N-(4-oxybutyl)-1,4-diazabicyclo[2.2.2]octyl-N′-acetate];

lup-20(29)-ene-3,28-bis[N-(1,4-diazabicyclo[2.2.2]octyl)acetate];

lup-20(29)-ene-3,28-bis[N-(N′-benzyldiazabicyclo[2.2.2]octyl)acetate);

lup-20(29)-ene-3,28-bis[N-(N′-(4-oxybutyl)diazabicyclo[2.2.2]octyl)acetate];

lup-20(29)-ene-3-[N-(1,4-diazabicyclo[2.2.2]octyl)acetate];

lup-20(29)-ene-3,28-bis[(tetramethylethylenediamine-N-yl)acetate];

lup-20(29)-ene-3,28-bis[(N′-benzyl-N,N,N′,N′-tetramethylethylenediamine-N-yl)acetate];

lup-20(29)-ene-3-[N-(N′-(benzyl)diazabicyclo[2.2.2]octyl)acetate];

bis(N,N′-pyridinium-2-ethyl)lup-20(29)-ene-3,28-dicarbamate;

1-(3,28-(diacetoxy)lup-20(29)-ene-30-yl)-4-(dimethylamino)pyridinium;

lup-20(29)-ene-3,28-bis(N-pyridinium-2-propionate);

lup-20(29)-ene-3,28-bis(N-pyridinium-3-propionate);

lup-20(29)-ene-3,28-bis(N-pyridinium-4-butyrate);

lup-20(29)-ene-3,28-bis(N-pyridinium-4-butyrate);

lup-20(29)-ene-3,28-bis(N-pyridinium-2-butyrate);

1-[3,28-(diacetoxy)lup-20(29)-ene-30-yl]-1,4-diazabicyclo[2.2.2]octyl;

3,28-bis[3-(1-piperidinyl)propanoyloxy]lup-20(29)-ene;

1-(3,28-dihydroxylup-20(29)ene-30-yl)-4-(dimethylamino)pyridinium;

lup-20(29)-ene-3,28-bis[N-(4-dimethylaminopyridinium)-2-propionate];

lup-20(29)-ene-3,28-bis[N-(1,4-diazabicyclo[2.2.2]octyl)-2-propionate];

1-(lup-20(29)-ene-30-yl)-1,4-diazabicyclo[2.2.2]octane;

1-(3,28-dihydroxylup-20(29)-ene-30-yl)-pyridinium; lup-20(29)-ene-3,28-bis[N-(1,4-diazabicyclo[2.2.2]octyl)-4-butyrate];

1-(3,28-dihydroxylup-20 (29)-ene-30-yl)-[N-3-(hydroxymethyl)pyridinium];

1-(3,28-dihydroxylup-20(29)-ene-30-yl)-[N-(3,5-dimethylpyridinium)];

bis[N-(1,4-diazabicyclo[2.2.2]octyl)-2-ethyl]-lup-20(29)ene-3,28-dicarbamate;

lup-20(29)-ene-3,28-bis[N-(3-oxymethylpyridinium)acetate];

lup-20(29)-ene-3,28-bis[N-(2-oxymethylpyridinium)acetate];

lup-20(29)-ene-3,28-bis[N-(2-methylureapyridinium)acetate];

lup-20(29)-ene-3-[N-(2-oxymethylpyridinium)acetate];

lup-20(29)-ene-3,28-bis[N-(N-methylmorpholino)acetate];

lup-20(29)-ene-3,28-bis[N-(4-hydroxyl-N-methylpiperidino)acetate];

lup-20 (29) -ene-3-[N-(3-ureamethylpyridinium)acetate];

lup-20(29)-ene-3-(N-pyridiniumacetate);

lup-20(29)-ene-3,28-bis[N-(1,4-diazabicyclo[2.2.2]octyl)-2-butyrate];

lup-20(29)-ene-3,28-bis[N-(4-dimethylpyridinium)-2-butyrate];

lup-20(29)-ene-3,28-bis[N-(4-dimethylaminopyridinium)-4-butyrate];

lup-20(29)-ene-3,28-bis[N-(4-dimethylaminopyridinium)-3-propionate];

1-(3,28-dihydroxylup-20(29)-ene-30-yl)-4-(hydroxymethyl)pyridinium;

1-(3,28-dihydroxylup-20(29)-ene-30-yl)-3-hydroxy-1-azabicyclo[2.2.2]octane;

lup-20(29)-ene-3,28-bis[N-(2,4-dimethylpyridinium)acetate];

lup-20(29)-ene-3,28-bis[N-(3,5-dimethylpyridinium)acetate];

lup-20(29)-ene-3,28-bis[N-(4-dimethylaminopyridinium)acetate];

lup-20(29)-ene-3-[N-(2-methylpyridinium)acetate];

lup-20(29)ene-3-[N-(2,4-dimethylpyridinium)acetate];

lup-20(29)-ene-3-[N-(4-hydroxy-N-methylpiperidino)acetate];

lup-20(29)-ene-3-[N-(N-methylmorpholino)acetate];

lup-20(29)-ene-3-[N-(3,5-dimethylpyridinium)acetate];

lup-20 (29) -ene-3-[N-(4-dimethylaminopyridinium)acetate];

lup-20 (29) -ene-3,28-bis(octyldimethylammoniumacetate);

lup-20(29)-ene-3-octyldimethylammoniumacetate;

lup-20(29)-ene-3,28-bis(tetradecyldimethylammoniumacetate);

lup-20(29)-ene-3-tetradecyldimethylammoniumacetate;

N,N,N′,N′-tetramethylethylenediamine-N,N′-bis-[lup-20(29)-ene-3-acetate];

1-[(lup-20(29)-en-3-yl)oxycarbonylmethyl]-4-aza-1-azonia-bicyclo[2.2.2]octane;

1-[(lup-20(29)-en-3-yl)oxycarbonylmethyl]trimethylammonium;

1-[(lup-20(29)-en-3-yl)oxycarbonylmethyl]pyridinium; or a combination thereof.
[41] The present invention also includes the composition of embodiment [1], wherein the triterpene is a compound of formula (V): 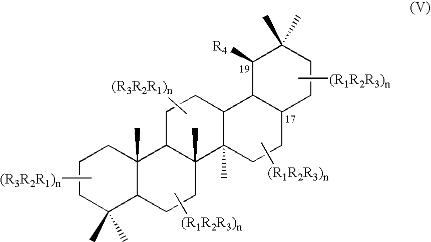
   a metabolite thereof, or a pro-drug thereof, or a pharmaceutically acceptable salt thereof wherein

each R₁ is independently absent, oxy, thio, or imino;

each R₂ is independently absent or alkylene;

each R₃ is independently hydrogen, N⁺-containing heteroaryl, N⁺-containing heterocycle, or —N⁺RₐR_{b}R_{c}; provided at least one R₃ is N⁺-containing heteroaryl, N⁺-containing heterocycle, or —N⁺RₐR_{b}R_{c};

R₄ is hydrogen, alkyl, or hydroxyalkyl;

or R₄ together with one R₁R₂R₃ forms a —OCH₂-bridging carbons 19 and 17;

wherein Rₐ, R_{b}, and R_{c} are each independently (C₁-C₂₄)alkyl, aryl, arylalkyl, heteroarylalkly, heterocycle, or heterocylealkyl;

wherein each n is independently 0-4, provided at least one n is not 0;

wherein any heteroaryl, heterocycle, or Rₐ, R_{b}, or R_{c} of R₃ can optionally be substituted on carbon with one or more alkyl, hydroxyalkyl, arylalkyl, heteroarylalkyl, aryl, heterocycle, heterocyclealkyl, oxo, hydroxy, halo, nitro, cyano, (C₁-C₆)alkoxy, trifluoromethyl, —COOR_{d}, —NR_{d}Rₑ, or cycloalkylalkyl;

wherein any cycloalkylalkyl can optionally be substituted on carbon with one or more hydroxyl, N⁺-containing heteroaryl, N⁺-containing heterocycle, —N⁺RₐR_{b}R_{c}, N⁺-containing heteroarylalkyloxy, N⁺-containing heterocyclealkyloxy, or —N⁺RₐR_{b}R_{c}oxy;

wherein R_{d} and Rₑ are each independently hydrogen or alkyl;

wherein any alkyl or alkylene of R₃ can optionally be substituted on carbon with one or more oxo, hydroxy, halo, nitro, cyano, (C₁-C₆)alkoxy, trifluoromethyl, —COOR_{d}, or —NR_{d}Rₑ, and optionally interrupted on carbon with one or more oxy, imino, or thio, and is optionally partially unsaturated.
[42] The present invention also includes the composition of embodiment [1], wherein the triterpene is a compound of formula (VI): 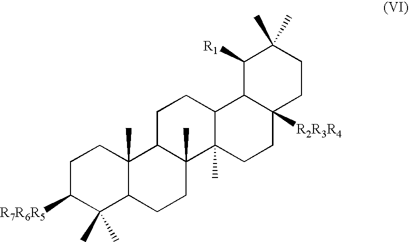
   wherein

R₁ is hydrogen, alkyl, or hydroxyalkyl,

R₂ is oxymethylene, thiomethylene, iminomethylene, or methylene;

R₃ and R₆ are each independently absent or alkylene;

R₄ and R₇ are each independently hydrogen, N⁺-containing heteroaryl, N⁺-containing heterocycle, or —NRₐR_{b}R_{c}; provided at least one of R₄ and R₇ is N⁺-containing heteroaryl, N⁺-containing heterocycle, —NRₐR_{b}R_{c}; or R₁, R₂, R₃, and R₄ are together —O—C(═X)—; wherein X is two hydrogens, oxo, or thioxo (═S);

wherein Rₐ, R_{b}, and R_{c} are each independently (C₁-C₂₄)alkyl, aryl, arylalkyl, heteroarylalkyl, heterocycle, or heterocylealkyl;

wherein R₅ is absent, oxy, thio, or imino;

wherein any heteroaryl, heterocycle, or Rₐ, R_{b}, or R_{c} of R₄ and R₇ can optionally be substituted on carbon with one or more alkyl, hydroxyalkyl, arylalkyl, heteroarylalkyl, aryl, heterocycle, heterocyclealkyl, oxo, hydroxy, halo, nitro, cyano, (C₁-C₆)alkoxy, trifluoromethyl, —COOR_{d}, —NR_{d}Rₑ, or cycloalkylalkyl;

wherein any cycloalkylalkyl can optionally be substituted on carbon with one or more hydroxyl, N⁺-containing heteroaryl, N⁺-containing heterocycle, —N⁺RₐR_{b}R_{c}, N⁺-containing heteroarylalkyloxy, N⁺-containing heterocyclealkyloxy, or —N⁺RₐR_{b}R_{c}oxy;

wherein R_{d} and Rₑ are each independently hydrogen or alkyl;

wherein any alkyl or alkylene of R₃, R₄, R₆, or R₇ can be optionally substituted on carbon with one or more oxo, hydroxy, halo, aryl, nitro, cyano, (C₁-C₆)alkoxy, trifluoromethyl, COOR_{d}, or —NR_{d}Rₑ, and optionally interrupted on carbon with one or more oxy, imino, or thio, and is optionally partially unsaturated;

or an acceptable salt thereof.
[43] The present invention also includes the composition of embodiment [42], wherein:

R₁ is hydrogen, alkyl, or hydroxyalkyl,

R₂ is oxymethylene, thiomethylene, iminomethylene, or methylene;

R₃ and R₆ are each independently absent or (C₁-C₅) alkylenecarbonyl;

R₄ and R₇ are each independently hydrogen, N-diazabicyclo[2.2.2]octyl; N-pyridinium; N-alkyl-N-piperidino; N-alkyl-N-morpholino; N-azabicyclo[2.2.2]octyl; or NRₐR_{b}R_{c};

or R₁, R₂, R₃, and R₄ are together —O—CH₂—;

wherein N-diazabicyclo[2.2.2]octyl; N-pyridinium; N-alkyl-N-piperidino; N-alkyl-N-morpholino; and N-azabicyclo[2.2.2]octyl can optionally be substituted on carbon with one or more alkyl, hydroxyalkyl, hydroxy, COOR_{d}, or NR_{d}Rₑ;

wherein Rₐ, R_{b}, and R_{c} are each independently aryl or (C₁-C₂₄)alkyl; wherein R_{d} and Rₑ are each independently hydrogen or alkyl;

wherein any alkylene or alkyl can optionally be substituted on carbon with one or more oxo, hydroxy, halo, nitro, cyano, trifluoromethyl, COOR_{d}, or —NR_{d}Rₑ, and optionally interrupted with one or more oxy, imino, or thio, and where any alkyl or alkylene can optionally be partially unsaturated.
[44] The present invention also includes the composition of embodiment [42], wherein R₁, R₂, R₃, and R₄ are together —O—CH₂—.
[45] The present invention also includes the composition of embodiment [42], wherein R₅ is oxy.
[46] The present invention also includes the composition of embodiment [42], wherein R₆ is acetyl.
[47] The present invention also includes the composition of embodiment [42], wherein R₇ is N-diazabicyclo[2.2.2]octyl; N-pyridinium; or —N⁺(CH₃)₃.
[48] The present invention also includes the composition of embodiment [42], wherein the cation of the compound is 1-[(19,28-epoxy-18-oleanan-3-yl)oxycarbonylmethyl]-4-aza-1-azonia-bicyclo(2.2.2]octane; [(19,28-epoxy-18-oleanan-3-yl)oxyqarbonylmethyl]trimethylammonium; or 1-[(19,28-epoxy-18-oleanan-3-yl)oxycarbonylmethyl]pyridinium.
[49] The present invention also includes the composition of any one of embodiments [1] to [48], wherein the triterpene, metabolite thereof, or pro-drug thereof is present up to about 30 wt. % of the composition.
[50] The present invention also includes the composition of any one of embodiments [1] to [49], wherein the triterpene, metabolite thereof, or pro-drug thereof is present up to about 20 wt. % of the composition.
[51] The present invention also includes the composition of any one of embodiments [1] to [49], wherein the triterpene, metabolite thereof, or pro-drug thereof is present up to about 10 wt. % of the composition.
[52] The present invention also includes the composition of any one of embodiments [1] to [49], wherein the triterpene, metabolite thereof, or pro-drug thereof is present up to about 5 wt. % of the composition.
[53] The present invention also includes the composition of any one of embodiments [1] to [49], wherein the triterpene, metabolite thereof, or pro-drug thereof is present in a concentration of about 0.001 μg/ml to about 5000 μg/ml.
[54] The present invention also includes the composition of any one of embodiments [1] to [49], wherein the triterpene, metabolite thereof, or pro-drug thereof is present in a concentration of about 12.5 μg/ml to about 1000 μg/ml.
[55] The present invention also includes the composition of any one of embodiments [1] to [54], wherein the lanolin, anhydrous lanolin, acetylated lanolin, lanosterol (lonosta-8,24-dien-3-ol), lanoceric acid, lanocerin, lanocerina, lanochol, lanolin alcohol, lanofier, lanofin, lanogel, lanogene, lanolate, lanolized A, lanolized RC, lanolized WW, liquid lanolin, lanosan, lanosol, lanotex 730, lanoxal 75, lanoxide 52, lanoxide 59, lanoxyl 30, lanpol, lanpolamide, lantox, lantox 55, lantrol, lantrol AWS, lanum, lanotex 730, lanoxal 75, lanoxide 52, lanoxide 59, lanoxyl 30, lanpol, lanidrol, lanigen, laniol, lanisolate, lanpolamide, laneto WWA, lanexol, lanfrax, petrolatum, white petrolatum, mineral oil, ceresin, panthenol, glycerin, polyethylene glycol (PEG), bisabolol, or combination thereof is present in a total amount of up to about 90 wt. % of the composition.
[56] The present invention also includes the composition of any one of embodiments [1] to [54], wherein the lanolin, anhydrous lanolin, acetylated lanolin, lanosterol (lonosta-8,24-dien-3-ol), lanoceric acid, lanocerin, lanocerina, lanochol, lanolin alcohol, lanofier, lanofin, lanogel, lanogene, lanolate, lanolized A, lanolized RC, lanolized WW, liquid lanolin, lanosan, lanosol, lanotex 730, lanoxal 75, lanoxide 52, lanoxide 59, lanoxyl 30, lanpol, lanpolamide, lantox, lantox 55, lantrol, lantrol AWS, lanum, lanotex 730, lanoxal 75, lanoxide 52, lanoxide 59, lanoxyl 30, lanpol, lanidrol, lanigen, laniol, lanisolate, lanpolamide, laneto WWA, lanexol, lanfrax, petrolatum, white petrolatum, mineral oil, ceresin, panthenol, glycerin, polyethylene glycol (PEG), bisabolol, or combination thereof is present in a total amount of up to about 80 wt. % of the composition.
[57] The present invention also includes the composition of any one of embodiments [1] to [54], wherein the lanolin, anhydrous lanolin, acetylated lanolin, lanosterol (lonosta-8,24-dien-3-ol), lanoceric acid, lanocerin, lanocerina, lanochol, lanolin alcohol, lanofier, lanofin, lanogel, lanogene, lanolate, lanolized A, lanolized RC, lanolized WW, liquid lanolin, lanosan, lanosol, lanotex 730, lanoxal 75, lanoxide 52, lanoxide 59, lanoxyl 30, lanpol, lanpolamide, lantox, lantox 55, lantrol, lantrol AWS, lanum, lanotex 730, lanoxal 75, lanoxide 52, lanoxide 59, lanoxyl 30, lanpol, lanidrol, lanigen, laniol, lanisolate, lanpolamide, laneto WWA, lanexol, lanfrax, petrolatum, white petrolatum, mineral oil, ceresin, panthenol, glycerin, polyethylene glycol (PEG), bisabolol, or combination thereof is present in a total amount of up to about 70 wt. % of the composition.
[58] The present invention also includes the composition of any one of embodiments [1] to [54], wherein the lanolin, anhydrous lanolin, acetylated lanolin, lanosterol (lonosta-8,24-dien-3-ol), lanoceric acid, lanocerin, lanocerina, lanochol, lanolin alcohol, lanofier, lanofin, lanogel, lanogene, lanolate, lanolized A, lanolized RC, lanolized WW, liquid lanolin, lanosan, lanosol, lanotex 730, lanoxal 75, lanoxide 52, lanoxide 59, lanoxyl 30, lanpol, lanpolamide, lantox, lantox 55, lantrol, lantrol AWS, lanum, lanotex 730, lanoxal 75, lanoxide 52, lanoxide 59, lanoxyl 30, lanpol, lanidrol, lanigen, laniol, lanisolate, lanpolamide, laneto WWA, lanexol, lanfrax, petrolatum, white petrolatum, mineral oil, ceresin, panthenol, glycerin, polyethylene glycol (PEG), bisabolol, or combination thereof is present in a total amount of up to about 60 wt. % of the composition.
[59] The present invention also includes the composition of any one of embodiments [1] to [58], that further includes water.
[60] The present invention also includes the composition of any one of embodiments [1] to [59], that further includes at least one of an ointment base, a humectant, and an emollient.
[61] The present invention also includes the composition of embodiment [1] that includes:

(a) a triterpene, a metabolite thereof, or a pro-drug thereof; and

(b) a substance selected from the group of lanolin, anhydrous lanolin, acetylated lanolin, lanosterol (lonosta-8,24-dien-3-ol), lanoceric acid, lanocerin, lanocerina, lanochol, lanolin alcohol, lanofier, lanofin, lanogel, lanogene, lanolate, lanolized A, lanolized RC, lanolized WW, liquid lanolin, lanosan, lanosol, lanotex 730, lanoxal 75, lanoxide 52, lanoxide 59, lanoxyl 30, lanpol, lanpolamide, lantox, lantox 55, lantrol, lantrol AWS, lanum, lanotex 730, lanoxal 75, lanoxide 52, lanoxide 59, lanoxyl 30, lanpol, lanidrol, lanigen, laniol, lanisolate, lanpolamide, laneto WWA, lanexol, lanfrax, petrolatum, white petrolatum, mineral oil, or combination thereof.
[62] The present invention also includes the composition of any one of embodiments [1] to [61], that further includes an absorption enhancer.
[63] The present invention also includes the composition of embodiment [62], wherein the absorption enhancer includes at least one of water, methanol, ethanol, 2-propanol, dimethyl sulfoxide, decylmethyl sulfoxide, tetradecyl methyl sulfoxide, 2-pyrrolidone, N-methyl-2-pyrrolidone, N-(2-hydroxyethyl) pyrrolidone, laurocapram, acetone, dimethyl acetamide, dimethyl formamide, tetrahydrofurfuryl alcohol, docusate sodium, sodium lauryl sulfate, quaternary ammonium salt, lecithin, cephalin, alkylbetamine, monglyceride, diglycxeride, triglyceride, lauryl alcohol, cetyl alcohol, stearyl alcohol, sucrose, sorbitan, polyethylene glycol, urea, and N,N-diethyl-m-toluamide.
[64] The present invention also includes the composition of any one of embodiments [1] to [63], that further includes a polyhydric alcohol selected from the group of glycerin, ethylene glycol, polyethylene glycol, propylene glycol, triethylene glycol, tetraethylene glycol, sorbitol, and combinations thereof.
[65] The present invention also includes the composition of any one of embodiments [1] to [64], that further includes a skin protectant selected from the group of aloe, glycerin, calamine, Vitamin E, Vitamin E acetate, Vitamin C, allantoin, aluminum hydroxide gel, bismuth subnitrate, boric acid, calamine, cocoa butter, dimethicone, glycerin, kaolin, live yeast cell derivative, petrolatum, pyridoxine hydrochloride, shark liver oil, sodium bicarbonate, sulfur, tannic acid, topical starch, mineral oil, ceresin, bisabolol, panthenol, trolamine, white petrolatum, zinc acetate, zinc carbonate zinc oxide, zinc sulfate, wax, beeswax, and combinations thereof.
[66] The present invention also includes the composition of any one of embodiments [1] to [65], that further includes an anti-infective agent selected from the group of:

[1R-(1R*, 3S*, 5R*, 6R*, 9R*, 11R*, 15S*, 16R*, 17R*, 18S*, 19E, 21E, 23E, 25E, 27E, 29E, 31E, 33R*, 35S*, 36R*, 37S*)]-33-[(3-Amino-3,6-dideoxy-β-D-mannopyranosyl)oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid (Amphotericin B);

5-fluorocytosine (Flucytosine);

2,4-difluoro-α,α¹-bis(1H-1,2,4-triazol-1-ylmethyl) benzyl alcohol) (Fluconazole);

griseofulvin microsize (Griseofulvin);

(E) -N-(6,6-dimethyl-2-hepten-4-ynyl)-N-methyl-1-naphthalenemethanamine hydrochloride) (Terbinafine);

cis-1-acetyl-4-[4-[(2-(2,4-dichlorophenyl)-2-(1H-imadazol-1-ylmethyl)-1,3-dioxolan-4-yl]methoxyl]phenyl]piperazine (Ketoconazole);

(±)-1-[(R*)-sec-butyl]-4-[p-[4-[p-[[(2R*,4S*)-2-(2,4-dichlorophenyl)-2-(1H-1,2,4-triazol-1-ylmethyl)-1,3-dioxolan-4-yl]methyoxy]phenyl]-1-piperazinyl]phenyl]-Δ²-1,2,4-triazolin-5-one mixture with (±)-1-[(R*)-sec-butyl]-4-[p-[4-[p-[[(2S*, 4R*)-2-(2,4-dichlorophenyl)-2-(1H-1,2,4-triazol-1-ylmethyl)-1,3-dioxolan-4-yl]methoxy]phenyl]-1-piperazinyl]phenyl]-Δ²-1,2,4-triazolin-5-one or (±)-1-[(RS)-sec-butyl]-4-[p-[4-[p-[[(2R, 4S)-2-(2,4-dichlorophenyl)-2-(1H-1,2,4-triazol-1-ylmethyl)-1,3-dioxolan-4-yl]-methoxy]phenyl]-1-piperazinyl]phenyl]-Δ²-1,2,4-triazolin-5-one (Itraconazole);

2-chloro-5-hydroxy-1,3-dimethylbenzene (Chloroxylenol);

griseofulvin ultramicrosize (Griseofulvin);

(E)-N-(6,6,-dimethyl-2-hepten-4-ynyl)-N-methyl-1-naphthalenemanamine hydrochloride (Terbinafine);

6-cyclohexyl-1-hydroxy-4-methyl-2(1H)-pyridinone (Ciclopirox);

N-4-tert-butyl-benzyl-N-methyl-1-naphthalenemethylamine hydrochloride (Butenafine hydrochloride);

nystatin;

(E)-N-(Cinnamyl-N-methyl-1-naphthalenemethylamine hydrochloride (Naftifine hydrochloride);

2′,4′-dichloro-2-imidazol-1-ylacetophenone (Z)-[0-(2,4-dichlorobenzyl)oxime]mononitrate (Oxiconazole nitrate),

6-cyclohexyl-1-hydroxy-4-methyl-2(1H)-pyridone (Ciclopirox);

selenium sulfide;

(±)-1-[4-(p-chlorophenyl)-2-[(2,6-dichlorophenyl)thio]butyl]imidazole mononitrate (Butoconazole nitrate);

([1-(o-chloro-.,.-diphenylbenzyl)imidazole]) (Clotrimazole);

(cis-1-[p-[[2-(2,4-dichlorophenyl)-2-(1H-1,2,4-triazol-1-ylmethyl)-1,3-dioxolan-4-yl]methoxy phenyl]-4-isopropyl-piperazine (Tercanazole);

6-cyclohexyl-1-hydroxy-4-methyl-2(1H)-pyridone (ciclopirox);

and combinations thereof.
[67] The present invention also includes the composition of any one of embodiments [1] to [66], that is formulated as an anti-fungicidal composition and further includes a fungicidal excipient.
[68] The present invention also includes the composition of any one of embodiments [1] to [66], that is formulated as a cosmetic composition and further includes a cosmetic excipient.
[69] The present invention also includes the composition of any one of embodiments [1] to [68], that is formulated for topical administration.
[70] The present invention also includes the composition of any one of embodiments [1] to [69], which is a cream.
[71] The present invention also includes the composition of any one of embodiments [1] to [69], which is a gel.
[72] The present invention also includes the composition of any one of embodiments [1] to [69], which is an ointment.
[73] The present invention also includes the composition of any one of embodiments [1] to [69], which is a lotion.
[74] The present invention also includes a composition that includes:

(a) betulin, a metabolite thereof, or a pro-drug thereof; and

(b) lanolin, anhydrous lanolin, acetylated lanolin, lanosterol (lonosta-8,24-dien-3-ol), lanoceric acid, lanocerin, lanocerina, lanochol, lanolin alcohol, lanofier, lanofin, lanogel, lanogene, lanolate, lanolized A, lanolized RC, lanolized WW, liquid lanolin, lanosan, lanosol, lanotex 730, lanoxal 75, lanoxide 52, lanoxide 59, lanoxyl 30, lanpol, lanpolamide, lantox, lantox 55, lantrol, lantrol AWS, lanum, lanotex 730, lanoxal 75, lanoxide 52, lanoxide 59, lanoxyl 30, lanpol, lanidrol, lanigen, laniol, lanisolate, lanpolamide, laneto WWA, lanexol, lanfrax, petrolatum, white petrolatum, mineral oil, ceresin, panthenol, glycerin, polyethylene glycol (PEG), bisabolol, or a combination thereof.
[75] The present invention also includes a composition that includes betulin, a metabolite thereof, or a pro-drug thereof; and VASELINE® Petroleum Jelly or K-Y® petroleum jelly.
[76] The present invention also includes a composition that includes betulin, a metabolite thereof, or a pro-drug thereof; and petroleum jelly.
[77] The present invention also includes a composition that includes betulin, a metabolite thereof, or a pro-drug thereof; and AQUAPHOR® ointment or EUCERIN® cream.
[78] The present invention also includes a composition that includes a triterpene, a metabolite thereof, or a pro-drug thereof; petrolatum; and an excipient.
[78] The present invention also includes a therapeutic method for treating a mammal afflicted with a fungal infection or a yeast infection. The method includes administering to a mammal in need of such treatment or at risk thereof, an effective anti-fungal amount of a composition of any one of embodiments [1] to [77].
[79] The present invention also includes the method of embodiment [78], wherein the mammal is a human.
[80] The present invention also includes the method of embodiment [78], wherein the fungal infection is caused by a dermatophytic fungus.
[81] The present invention also includes the method of embodiment [80], wherein the dermatophytic fungus is Microsporum canis, Microsporum gypseum, Microsporum audouinii, Trichophyton tonsurans, Trichophyton mentagrophytes, Epidermophyton floccosum, Trichophyton rubrum, or Pityrosporum ovale.
[82] The present invention also includes the method of embodiment [78], wherein the fungal infection is caused by Candida albicans or Candida guilliermoundi.
[83] The present invention also includes the method of embodiment [78], wherein the fungal infection is caused by Blastomyces dermatidis or Cryptococcus neoformans.
[84] The present invention also includes the method of embodiment [78], wherein the fungal infection is present on a nail of the mammal, under the nail of the mammal, or a combination thereof.
[85] The present invention also includes the method of embodiment [78], wherein the fungal infection is present on a toe-nail of the mammal, under the toe-nail of the mammal, or a combination thereof.
[86] The present invention also includes the method of embodiment [78], wherein the fungal infection is present on the scalp of the mammal.
[87] The present invention also includes the method of embodiment [78], wherein the fungal infection or yeast infection is present on the vagina of the mammal, in the vagina of the mammal, or a combination thereof.
[88] The present invention also includes the method of embodiment [78], wherein the fungal infection or yeast infection is present on a skin surface of the mammal.
[89] The present invention also includes the method of embodiment [78], wherein the triterpene, metabolite thereof, or pro-drug thereof is present in about 0.1 μg to about 500 mg of the composition.
[90] The present invention also includes the method of embodiment [78], wherein the triterpene, metabolite thereof, or pro-drug thereof is present in a concentration of about 0.001 μg/ml to about 5000 μg/ml.
[91] The present invention also includes the method of embodiment [78], wherein the triterpene, metabolite thereof, or pro-drug thereof is present in a concentration of about 12.5 μg/ml to about 1000 μg/ml.
[92] The present invention also includes a method of inhibiting or killing a fungus or a yeast. The method includes contacting the fungus or yeast with an effective anti-fungal or ant-yeast amount of a composition of any one of embodiments [1] to [77].
[93] The present invention also includes the method of embodiment [92], wherein the contacting is in vitro.
[94] The present invention also includes the method of embodiment [92], wherein the contacting is in vivo.
[95] The present invention also includes the method of embodiment [93], wherein the fungal infection is present on plant tissue.
[96] The present invention also includes the method of embodiment [92], wherein the fungus is present on turf grass.
[97] The present invention also includes the method of embodiment [92], wherein the fungus causes the disease dollar spot or brown patch.
[98] The present invention also includes the method of embodiment [95], wherein the plant tissue includes bark, roots, leaves, flowers, needles, bulbs, berries, rhizomes, rootstocks, stems, seeds, or any combination thereof.
[99] The present invention also includes the method of embodiment [92], wherein the triterpene, metabolite thereof, or pro-drug thereof is present in about 0.1 μg to about 500 mg of the composition.
[100] The present invention also includes the method of embodiment [92], wherein the triterpene, metabolite thereof, or pro-drug thereof is present in a concentration of about 0.001 μg/ml to about 5000 μg/ml.
[101] The present invention also includes the method of embodiment [92], wherein the triterpene, metabolite thereof, or pro-drug thereof is present in a concentration of about 12.5 μg/ml to about 1000 μg/ml.
[102] The present invention also includes a method for enhancing skin appearance. The method includes administering to a skin surface in need of the appearance enhancement a cosmetically effective amount of a composition of any one of embodiments [1] to [77].
[103] The present invention also includes the method of embodiment (102], wherein the skin surface in need of the appearance enhancement is afflicted with a fungal infection or a yeast infection.
[104] The present invention also includes the method of embodiment [102], wherein the skin surface in need of the appearance enhancement is afflicted with a fungal infection, a yeast infection, cracked skin, dry skin, damaged skin, sunburned skin, skin with a minor burn, irritated skin, skin with a rash, chapped skin, raw skin, skin afflicted with Dermatitis, skin with an abrasion, skin associated with Atopic Dermatitis, skin associated with laser resurfacing, skin associated with chemical peels, skin associated with radiation therapy, skin afflicted with acne and treated with Accutane® (Isotretinoin), skin afflicted with erythema, skin afflicted with fissuring, skin with a diaper rash, or a combination thereof.
[105] The present invention also includes a method for treating skin afflicted with a condition selected from the group of: a fungal infection, a yeast infection, cracked skin, dry skin, damaged skin, sunburned skin, skin with a minor burn, irritated skin, skin with a rash, chapped skin, raw skin, skin afflicted with Dermatitis, skin with an abrasion, skin associated with Atopic Dermatitis, skin associated with laser resurfacing, skin associated with chemical peels, skin associated with radiation therapy, skin afflicted with acne and treated with Accutane® (Isotretinoin), skin afflicted with erythema, skin afflicted with fissuring, skin with a diaper rash, or a combination thereof. The method includes dministering to a skin surface in need of the treatment or at risk thereof, a composition of any one of embodiments [l] to [77].
[106] The present invention also includes a method of inhibiting or killing a fungus or yeast. The method includes contacting the fungus or yeast with an effective anti-fungal or ant-yeast amount of a composition that includes petrolatum or white petrolatum, mineral oil, ceresin, panthenol, glycerin, polyethylene glycol (PEG), and bisabolol.
[107] The present invention also includes a method of inhibiting or killing a fungus or yeast. The method includes contacting the fungus or yeast with an effective anti-fungal or ant-yeast amount of a composition that includes petrolatum, mineral oil, ceresin and lanolin alcohol.
[108] The present invention also includes a method of inhibiting or killing a fungus or yeast. The method includes contacting the fungus or yeast with an effective anti-fungal amount or ant-yeast of a composition that includes VASELINE® Petroleum Jelly or K-Y® petroleum jelly.
[109] The present invention also includes a method of inhibiting or killing a fungus or yeast. The method includes contacting the fungus or yeast with an effective anti-fungal or ant-yeast amount of a composition that includes petroleum jelly.
[110] The present invention also includes a method of inhibiting or killing a fungus or a yeast. The method includes contacting the fungus or yeast with an effective anti-fungal or ant-yeast amount of a composition that includes AQUAPHOR® ointment.
[111] The present invention also includes a therapeutic method for treating a mammal afflicted with a fungal infection or a yeast infection. The method includes administering to a mammal in need of such treatment or at risk thereof, an effective anti-fungal amount or an effective anti-yeast amount of a composition that includes: betulin, a metabolite thereof, or a pro-drug thereof; and at least one of lanolin alcohol, petrolatum, white petrolatum, mineral oil, ceresin, panthenol, glycerin, polyethylene glycol (PEG), and bisabolol.
[112] The present invention also includes a therapeutic method for treating a mammal afflicted with a fungal infection or a yeast infection. The method includes administering to a mammal in need of such treatment or at risk thereof, an effective anti-fungal amount or an effective anti-yeast amount of a composition that includes: betulin, a metabolite thereof, or a pro-drug thereof; and at least one of AQUAPHOR® ointment, EUCERIN® cream, VASELINE® Petroleum Jelly, and K-Y® Petroleum Jelly.

The invention is further defined by reference to the following examples describing in detail the preparation of the compositions of the invention. It will be apparent to those skilled in the art that many modifications, both to materials and methods, may be practiced without departing from the purpose and interest of this invention.

### EXAMPLES

### Example 1

### Agar Dilution Method 1

Sabourand Dextrose Agar was prepared according the manufacturers instructions and 5 ml was dispensed in 100 X 25 mm tubes and autoclaved for 121° C. for 15 minutes. To each tube containing 5 ml of liquid agar at 45° C., different concentrations of test antifungal compound in DMSO was added, subsequently each was solidified in slants, and inoculated with fungal cultures. After incubation for 10-12 days at 25° C. to 28° C., the growth of the fungus was recorded every second day. The Minimum Inhibitory Concentration (MIC) was determined by the tube containing the lowest concentration of test compound which inhibited fungal growth. See, Table 1.

TABLE 1
Antifungal Activities of Betulin and Betulin
derivatives against Human Pathogenic Fungi

| | [ ] | Micro- | | | | | | | |
|---|---|---|---|---|---|---|---|---|---|
| Test | μg/ml | sporum | Micro- | | | | | | |
| Compounds | in DMSO | canis | sporum | Microsporum | Trichophyton | Trichophyton | Trichophyton | Epidermophyton | Pityrosporum ovale |
| Betulin | 100 | − | − | − | − | − | − | − | +/− |
| | 50 | +/− | − | ++ | ++ | + | +/− | − | ++ |
| | 25 | + | +/− | ++ | ++ | ++ | + | + | +++ |
| | 12.5 | ++ | +/− | +++ | +++ | ++ | ++ | + | +++ |
| Allobetulin | 75 | − | − | − | − | − | − | − | − |
| | 38 | ++ | − | ++ | ++ | + | + | + | ++ |
| | 19 | ++ | − | ++ | ++ | ++ | + | + | +++ |
| | 9 | +++ | +/− | +++ | +++ | ++ | ++ | ++ | +++ |
| Betulin 28- | 100 | − | − | − | − | − | − | − | − |
| phthalate | 50 | − | − | ++ | − | + | + | + | ++ |
| | 25 | + | + | ++ | + | + | ++ | ++ | +++ |
| | 12.5 | ++ | ++ | +++ | ++ | ++ | +++ | +++ | +++ |
| Allobetulin | 100 | − | − | − | − | − | − | − | − |
| 3-glutarate | 50 | − | − | ++ | − | + | + | + | ++ |
| | 25 | +/− | +/− | ++ | +/− | ++ | + | + | +++ |
| | 12.5 | +/− | + | +++ | + | ++ | ++ | ++ | +++ |
| Griseofulvin | 1000 | − | − | − | − | − | − | − | − |
| | 500 | − | − | − | − | − | − | − | ++ |
| | 250 | +/− | − | +/− | +/− | +/− | + | + | +++ |
| | 125 | +/− | − | +/− | +/− | +/− | ++ | ++ | +++ |
| Nystatin | 1000 | − | − | − | − | − | − | − | − |
| | 500 | − | − | − | − | +/− | − | − | − |
| | 250 | +/− | − | +/− | +/− | + | + | − | +/− |
| | 125 | + | − | + | + | + | + | +/− | + |
| DMSO | 0.5 | − | − | − | − | − | − | − | − |
| control | 0.25 | + | − | ++ | ++ | + | + | + | ++ |
| | 0.12 | + | + | ++ | ++ | + | + | + | +++ |
| | 0.062 | ++ | + | +++ | +++ | ++ | ++ | ++ | +++ |
| Fungi | | +++ | +++ | +++ | +++ | +++ | +++ | +++ | +++ |
| Control | | | | | | | | | |

+++ = Maximum Fungal Growth
++ = Moderate Fungal Growth
+ = Minimum Fungal Growth
+/− = Very little Fungal Growth
− = No Fungal Growth

### Example 2

### Agar Dilution Method 2

Sabourand Dextrose Agar was prepared according the manufacturers instructions and 5 ml was dispensed in 100×25 mm tubes and autoclaved for 121° C. for 15 minutes. To each tube containing 5 ml of liquid agar at 45° C., 4 mg/ml of test antifungal compound was added, subsequently each was solidified in slants, and inoculated with fungal cultures. After incubation for 10-12 days at 25° C. to 28° C., the growth of the fungus was recorded every second day. Table 2 contains the growth at the 12^{th} day of inoculation.

TABLE 2
Results using the Agar Dilution Method 2 in
concentration (μg/ml)

| Compound | M. canis | M. audouinii | T. tonsurans |
|---|---|---|---|
| Betulin | 50 | 25 | n/a |
| Allobetulin | 25 | 9 | 25 |
| 3-glutarate | | | |
| Betulin 28- | 50 | 25 | n/a |
| phthalate | | | |
| Griseofulvin | 250 | <125 | 250 |
| Nystatin | 250 | <125 | 250 |

### Example 3

### Anti-Fungal Activity Against Pityrosporum ovale

Betulin and its derivatives have low solubility in water, accordingly the stock solution of the test compounds were prepared in DMSO. The stock solutions for griseofulvin and nystatin were prepared in DMSO at a concentration of 10,000 μg/ml. The yeast Pityrosporum ovale (ATCC Rockville, Md.) was grown in Pityrosporum medium (ATCC Rockville, Md.) at 37° C. for 2-3 days. 5 ml of the medium was dispensed in 100×25 mm tubes and autoclaved at 121° C. for 15 minutes. To each tube at 45° C., 4 mg/ml of test compound was added and solidified in slants, subsequently, each was inoculated with P. ovale and incubated at 37° C. for 18 to 24 hours.

Allobetulin, allobetulin L-valine ester, betulin 28-glutarate, and betulin 3,28-diglutarate inhibited the growth at 4 mg/ml of test compound. The tests were comparable to that of nystatin against P. ovale infection which also inhibited at 4 mg/ml.

### Example 4

### Double Blind Experiment

A double blind study was conducted to determine the safety and efficacy of a betulin formulation applied topically to at least one toenail infected with dermatophyte toe onychomycosis. The study was conducted over six months wherein the subjects were evaluated at weeks 2, 6, 17, 26, and 31. At week 45, subjects came in for a post treatment follow-up visit. Sixty-six subjects, 15 males and 51 females ranging in age from 18 to 72 years, were tested during the study. Each subject had onychomycosis of one toe nail involving not more than 50% of at least one nail which was confirmed by microbiological sampling.

The subjects were randomly divided into 2 groups: Group A comprising 35 subjects that were tested with the Betulin formulation and Group B comprising 31 subjects that were tested using the Placebo formulation. The Betulin formulation contained betulin in Aquaphor while the Placebo formulation had no betulin.

Each group used the formulation twice daily (BID, AM and PM) for six months wherein application of the formulation was made to the base of the nail (nail bed) around the perimeter and to the affected nail plate. Subjects were also instructed to apply the formulation to adjacent nails, if these were affected.

Evaluations were conducted at Weeks 2, 6, 17, and 31. During each visit, fungal cultures and/or photographic documentation was collected. At Week 31, clinical confirmation of onychomycosis was obtained by dermatophyte culture. At Week 45, the follow up visit determined whether fungal dermatitis was present using directed microscopy and potassium hydroxide (KOH) staining.

Visual evaluations were conducted on the first week (Baseline) and Weeks 2, 16, 17, and 31. The evaluations including scaling, cracking, discoloration, erythema, and dryness. The results were expressed as the mean for each group and presented in Table 3 for Betulin formulation and Table 4 for the Placebo formulation. The scale used was 0=absent; 1=mild; 2=moderate; and 3=severe.

TABLE 3
Mean Visual Evaluation Scores for Betulin
formulation

| Parameter | Baseline | Week 2 | Week 6 | Week 17 | Week 31 |
|---|---|---|---|---|---|
| Scaling | 1.09 | 0.79^{a} | 0.56^{a} | 0.53^{a} | 0.24^{a} |
| Cracking | 0.09 | 0.09 | 0.06 | 0.18 | 0.06 |
| Discoloration | 1.5 | 1.41 | 1.06^{a} | 0.94^{a} | 0.91^{a} |
| Erythema | 0.03 | 0.06 | 0.06 | 0.00 | 0.00 |
| Dryness | 0.29 | 0.12 | 0.03^{a} | 0.15 | 0.00^{a} |

^{a}Statistically significant difference from baseline, p < 0.05.

TABLE 4
Mean Visual Evaluation Scores for Placebo
formulation

| Parameter | Baseline | Week 2 | Week 6 | Week 17 | Week 31 |
|---|---|---|---|---|---|
| Scaling | 1.14 | 0.9^{a} | 0.55^{a} | 0.45^{a} | 0.07^{a} |
| Cracking | 1.1 | 0.04 | 0.04 | 0.04 | 0.00 |
| Discoloration | 1.39 | 1.35 | 1.04^{a} | 1^{a} | 0.74^{a} |
| Erythema | 0.04 | 0.04 | 0.07 | 0.00 | 0.00 |
| Dryness | 0.66 | 0.1^{a} | 0.17^{a} | 0.1^{a} | 0.00^{a} |

^{a}Statistically significant difference from baseline, p < 0.05.

The results of the visual evaluation for the Betulin formulation showed no significant increase in erythema, no statistically significant change in nail cracking, and no decrease was observed in dryness (Weeks 6 and 31), scaling (all evaluations), and nail discoloration (Weeks 6, 17, and 31). The results of the visual evaluation for the Placebo formulation showed no significant change in erythema, no significant decrease in nail cracking, and a statistically significant decrease in dryness (all evaluations), scaling (all evaluations), and nail discoloration (Weeks 6, 17, and 31).

At the end of 31 weeks of treatment, both groups demonstrated a mycological cure by fungal culture. KOH evaluation 14 weeks after completion of the treatment showed a mycological cure in 7% of the betulin treated group and 11% of the placebo treated group. The population of fungal specimens decreased in all subjects compared with Baseline evaluations. After 17 weeks of treatment, 50% of betulin treated group and 38% of the placebo treated group achieved a visual cure, after 31 weeks, 62% and 56% showed a visual cure. A significantly greater number of betulin treated subjects showed a 2-level or greater visual improvement in nail discoloration at Week 17, over the placebo treated subjects.

### Example 5

### 48-Hour Patch Test

A study was conducted to determine the primary dermal irritation potential of a test article when applied to the skin of human subjects for 48 hours under an occlusive patch. The study was conducted over four days wherein the subjects were evaluated after 48 hours. At the end of 48-hours, the test patch was removed and the skin was evaluated and scored for dermal reactivity. A second reading was taken after 48 hours, i.e. 96 hours post-application. Fifty two subjects, 14 males and 38 females ranging in age from 18 to 69 years, were tested during the study.

A sufficient amount of 2% betulin in Aquaphor, an amount adequate to cover the surface of the patch unit, about 0.05 g, was placed onto ½″ analytical paper disc affixed to Blenderm® (3M) occlusive surgical tape. Subsequently, the test patch was applied to the inner forearm of each subject for a period of 48 hours. The test patch was reinforced with 2″ Scanpor (Allerderm) semi-occlusive surgical tape.

At the end of the 48 hour test period, the test patch was removed and the skin evaluated for dermal reactivity, followed by a second evaluation after another 48 hours. Dermal responses were evaluated using the following 6 point scale. 0=No evidence of any effect; +=Barely perceptible (minimal, faint, uniform or spotty erythema); 1=mild (pink, uniform erythema covering most of the contact site); 2=moderate (pink-red erythema uniform in the entire contact site); 3=marked (bright red erythema with or without petechiae or papules); and 4=severe (deep red erythema with or without vesiculation or weeping). No skin reactivity was observed in all test subjects during the time of the study.

### Example 6

### Repeated Patch Test

A study was conducted to determine the repeated dermal irritation potential of a test article when applied to the skin of human subjects for 24 hours under an occlusive patch. The study was conducted over several weeks wherein the subjects underwent an induction phase and after a two week rest a challenge phase. Forty eight subjects were tested, however, seven discontinued the test for reasons unrelated to the study.

The induction phase comprised applied a patch to the test subject for 24 hours. At the end of 24-hours, the test patch was removed and the skin was evaluated and scored for dermal reactivity. After a 24 hour rest period, the procedure was repeated for a total of three times per week for a total of nine applications.

A sufficient amount of 2% betulin in Aquaphor, an amount adequate to cover the surface of the patch unit, about 0.1 g to about 0.015 g, was placed onto a Parke-Davis Readi-Bandage® occlusive patch. The patch was applied to the back of each subject between the scapulae and waist, adjacent to the spinal midline. The subjects removed the patch after 24 hours and rested for another 24 hours prior to evaluation of the site. If a subject developed a positive reaction of a 2-level erythema or greater during the Induction phase, the patch was applied to a new site. If a second 2-level erythema reaction or greater occurred, no further applications were made.

After a rest period of two weeks, the subjects were tested using the Challenge patch. The Challenge patch was scored every 24 and 72 hours after application. All subjects were instructed to report any delayed skin reactivity that occurred after the final Challenge patch reading.

Dermal responses were evaluated using the following 6 point scale. 0=No evidence of any effect; +=Barely perceptible (minimal, faint, uniform or spotty erythema); 1=mild (pink, uniform erythema covering most of the contact site); 2=moderate (pink-red erythema uniform in the entire contact site); 3=marked (bright red erythema with or without petechiae or papules); and 4=severe (deep red erythema with or without vesiculation or weeping). Other observed dermal sequelae, e.g. edema, dryness, or hypo- or hyperpigmentation, were evaluated and scored as mild, moderate, or severe.

No skin reactivity was observed in all test subjects during the Induction phase of the study. During the Challenge phase, a barely perceptible (+) patch test response was observed on the subject at the 24 hour evaluation. By the 72 hour evaluation, however, no skin reactivity was observed.

What is claimed is:

1. A composition comprising:
(a) a triterpene selected from the group of betulin, betulinic acid, lupeol, betulin-3-caffeate, allobetulin, and combinations thereof; and
(b) a substance selected from the group of lanolin, anhydrous lanolin, acetylated lanolin, lanosterol (lonosta-8,24-dien-3-ol), lanoceric acid, lanocerin, lanocerina, lanochol, lanolin alcohol, lanofier, lanofin, lanogel, lanogene, lanolate, lanolized A, lanolized RC, lanolized WW, liquid lanolin, lanosan, lanosol, lanotex 730, lanoxal 75, lanoxide 52, lanoxide 59, lanoxyl 30, lanpol, lanpolamide, lantox, lantox 55, lantrol, lantrol AWS, lanum, lanotex 730, lanoxal 75, lanoxide 52, lanoxide 59, lanoxyl 30, lanpol, lanidrol, lanigen, laniol, lanisolate, lanpolamide, laneto WWA, lanexol, lanfrax, petrolatum and combinations thereof.

2. The composition of claim 1, wherein the triterpene is present up to about 5 wt. % of the composition.

3. The composition of claim 1, wherein the triterpene is present in a concentration of about 12.5 μg/ml to about 1000 μg/ml.

4. The composition of claim 1, that further comprises water.

5. The composition of claim 1, that further comprises an absorption enhancer.

6. The composition of claim 5, wherein the absorption enhancer includes at least one of water, methanol, ethanol, 2-propanol, dimethyl sulfoxide, decylmethyl sulfoxide, tetradecyl methyl sulfoxide, 2-pyrrolidone, N-methyl-2-pyrrolidone, N-(2-hydroxyethyl) pyrrolidone, laurocapram, acetone, dimethyl acetamide, dimethyl formamide, tetrahydrofurfuryl alcohol, docusate sodium, sodium lauryl sulfate, quaternary ammonium salt, lecithin, cephalin, alkylbetamine, monglyceride, diglycxeride, triglyceride, lauryl alcohol, cetyl alcohol, stearyl alcohol, sucrose, sorbitan, polyethylene glycol, urea, and N,N-diethyl-m-toluamide.

7. The composition of claim 1, that further comprises a polyhydric alcohol selected from the group of glycerin, ethylene glycol, polyethylene glycol, propylene glycol, triethylene glycol, tetraethylene glycol, sorbitol, and combinations thereof.

8. The composition of claim 1, that further comprises a skin protectant selected from the group of aloe, glycerin, calamine, Vitamin E, Vitamin E acetate, Vitamin C, allantoin, aluminum hydroxide gel, bismuth subnitrate, boric acid, calamine, cocoa butter, dimethicone, glycerin, kaolin, live yeast cell derivative, petrolatum, pyridoxine hydrochloride, shark liver oil, sodium bicarbonate, sulfur, tannic acid, topical starch, mineral oil, ceresin, bisabolol, panthenol, trolamine, white petrolatum, zinc acetate, zinc carbonate zinc oxide, zinc sulfate, wax, beeswax, methylchloroisothiazolinone, methylisothiazolinone and combinations thereof.

9. The composition of claim 1, that further comprises an anti-infective agent selected from the group of:
[1R-(1R*, 3S*, 5R*, 6R*, 9R*, 11R*, 15S*, 16R*, 17R*, 18S*, 19E, 21E, 23E, 25E, 27E, 29E, 31E, 33R*, 35S*, 36R*, 37S*)]-33-[(3-Amino-3,6-dideoxy-β-D-mannopyranosyl)oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid (Amphotericin B);
5-fluorocytosine (Flucytosine);
2,4-difluoro-α,α¹-bis(1H-1,2,4-triazol-1-ylmethyl) benzyl alcohol) (Fluconazole);
griseofulvin microsize (Griseofulvin);
(E)-N-(6,6-dimethyl-2-hepten-4-ynyl)-N-methyl-1-naphthalenemethanamine hydrochloride) (Terbinafine);
cis-1-acetyl-4-[4-[(2-(2,4-dichlorophenyl)-2-(1H-imadazol-1-ylmethyl)-1,3-dioxolan-4-yl]methoxyl]phenyl]piperazine (Ketoconazole);
(±)-1-[(R*)-sec-butyl]-4-[p-[4-[p-[[(2R*,4S*)-2-(2,4-dichlorophenyl)-2-(1H-1,2,4-triazol-1-ylmethyl)-1,3-dioxolan-4-yl]methyoxy]phenyl]-1-piperazinyl]phenyl]-Δ²-1,2,4-triazolin-5-one mixture with (±)-1-[(R*)-sec-butyl]-4-[p-[4-[p-[[(2S*, 4R*)-2-(2,4-dichlorophenyl)-2-(1H-1,2,4-triazol-1-ylmethyl)-1,3-dioxolan-4-yl]methoxy]phenyl]-1-piperazinyl]phenyl]-Δ²-1,2,4-triazolin-5-one or (±)-1-[(RS)-sec-butyl]-4-[p-[4-[p-[[(2R, 4S)-2-(2,4-dichlorophenyl)-2-(1H-1,2,4-triazol-1-ylmethyl)-1,3-dioxolan-4-yl]-methoxy]phenyl]-1-piperazinyl]phenyl]-Δ²-1,2,4-triazolin-5-one (Itraconazole);
2-chloro-5-hydroxy-1,3-dimethylbenzene (Chloroxylenol);
griseofulvin ultramicrosize (Griseofulvin);
(E)-N-(6,6,-dimethyl-2-hepten-4-ynyl)-N-methyl-1-naphthalenemanamine hydrochloride (Terbinafine);
6-cyclohexyl-1-hydroxy-4-methyl-2(1H)-pyridinone (Ciclopirox);
N-4-tert-butyl-benzyl-N-methyl-1-naphthalenemethylamine hydrochloride (Butenafine hydrochloride);
nystatin;
(E) -N-(Cinnamyl-N-methyl-1-naphthalenemethylamine hydrochloride (Naftifine hydrochloride);
2′,4′-dichloro-2-imidazol-1-ylacetophenone (Z)-[0-(2,4-dichlorobenzyl)oxime]mononitrate (Oxiconazole nitrate),
6-cyclohexyl-1-hydroxy-4-methyl-2(1H)-pyridone (Ciclopirox);
selenium sulfide;
(±)-1-[4-(p-chlorophenyl)-2-[(2,6-dichlorophenyl)thio]butyl]imidazole mononitrate (Butoconazole nitrate);
([1-(o-chloro-.,.-diphenylbenzyl) imidazole]) (Clotrimazole);
(cis-1-[p-[[2-(2,4-dichlorophenyl)-2-(1H-1,2,4-triazol-1-ylmethyl)-1,3-dioxolan-4-yl]methoxy phenyl]-4-isopropyl-piperazine (Tercanazole);
6-cyclohexyl-1-hydroxy-4-methyl-2(1H)-pyridone (ciclopirox);
and combinations thereof.

10. The composition of claim 1, that is formulated for topical administration.

11. The composition of claim 1, which is a cream.

12. The composition of claim 1, which is a gel.

13. The composition of claim 1, which is an ointment.

14. The composition of claim 1, which is a lotion.

15. A therapeutic method for treating a mammal afflicted with a fungal infection or a yeast infection, the method comprising administering to a mammal in need of such treatment or at risk thereof, an effective anti-fungal amount of a composition of claim 1.

16. The method of claim 15, wherein the mammal is a human.

17. The method of claim 15, wherein the fungal infection is caused by a dermatophytic fungus.

18. The method of claim 17, wherein the dermatophytic fungus is Microsporum canis, Microsporum gypseum, Microsporum audouinii, Trichophyton tonsurans, Trichophyton mentagrophytes, Epidermophyton floccosum, Trichophyton rubrum, or Pityrosporum ovale.

19. The method of claim 15, wherein the fungal infection is caused by Candida albicans or Candida guilliermoundi.

20. The method of claim 15, wherein the fungal infection is caused by Blastomyces dermatidis or Cryptococcus neoformans.

21. The method of claim 15, wherein the fungal infection is present on a nail of the mammal, under the nail of the mammal, or a combination thereof.

22. The method of claim 15, wherein the fungal infection is present on the scalp of the mammal.

23. A method of inhibiting or killing a fungus or a yeast, the method comprising contacting the fungus or yeast with an effective anti-fungal or ant-yeast amount of a composition of claim 1.

24. The method of claim 23, wherein the contacting is in vitro.

25. The method of claim 23, wherein the contacting is in vivo.

26. The method of claim 23, wherein the fungus is present on plant tissue.

27. The method of claim 23, wherein the fungus is present on turf grass.

28. A method for enhancing skin appearance in a mammal, the method comprising administering to a skin surface in need of the appearance enhancement a cosmetically effective amount of a composition of claim 1.

29. The method of claim 28, wherein the skin surface in need of the appearance enhancement is afflicted with a fungal infection or a yeast infection.

30. The method of claim 28, wherein the skin surface in need of the appearance enhancement is afflicted with a fungal infection, a yeast infection, a viral infection, a bacterial infection, cracked skin, dry skin, damaged skin, sunburned skin, skin with a minor burn, irritated skin, skin with a rash, chapped skin, raw skin, skin afflicted with dermatitis, skin with an abrasion, skin associated with atopic dermatitis, skin associated with laser resurfacing, skin associated with chemical peels, skin associated with radiation therapy, skin afflicted with acne and treated with Accutane® (Isotretinoin), skin afflicted with erythema, skin afflicted with fissuring, skin with a diaper rash, or a combination thereof.
